(19) United States
(12) Patent Application Publication
(10) Pub. No.: US 2025/0389714 A1
(43) Pub. Date: Dec. 25, 2025

(54) PHARMACEUTICAL COMPOSITION FOR TREATING DISEASES RELATED TO TAUOPATHY

(71) Applicant: SEOUL NATIONAL UNIVERSITY R&DB FOUNDATION, Seoul (KR)

(72) Inventors: Yong Keun JUNG, Seoul (KR); Youbin KIM, Yongin-si (KR)

(21) Appl. No.: 18/705,699

(22) PCT Filed: Oct. 26, 2022
(86) PCT No.: PCT/KR2022/016443

§ 371 Date: Mar. 27, 2025

(30) Foreign Application Priority Data
Oct. 27, 2021 (KR) 10-2021-0144795

(51) Int. Cl. G01N 33/50

(52) U.S. Cl. CPC G01N 33/5058; G01N 33/5023

(57) ABSTRACT

The present invention provides pharmaceutical composition for the treatment of tauopathy-related diseases that can significantly improve cognitive and behavioral impairments by reducing neuronal uptake and propagation of disease-associated tau.

### REFERENCE TO AN ELECTRONIC SEQUENCE LISTING

The contents of the electronic sequence listing (SPJ20245284US_SEQ: Size: 23,902 bytes; and Date of Creation: Apr. 24, 2024) is herein incorporated by reference in its entirety. The contents of the electronic sequence listing in no way introduces new matter into the specification.

### TECHNICAL FIELD

The present invention relates to a pharmaceutical composition for treating tauopathy-related diseases, wherein the pharmaceutical composition for treating tauopathy-related diseases effectively ameliorate tauopathies.

### BACKGROUND ART

The distribution of pathological tau inclusions widens during disease progression and strongly correlates with the clinical stage of tauopathies including Alzheimer's disease (AD) (H. Braak, et al., Acta Neuropathol . . . 82:239-259, 1991). Previous studies have focused on demonstrating synaptic transmission of various tau species in vitro and in vivo. Those have highlighted macropinocytosis-mediated tau internalization in neurons, which is highly mediated by heparan sulfate proteoglycans (HSPGs) (B. B. Holmes, et al., Proc. Natl. Acad. Sci. USA, 110: E3138-E3147, 2013). Low-density lipoprotein receptor-related protein 1 (LRP1) cooperates with HSPG to regulate tau entry into neurons, whereas its contribution to tau pathogenesis is undetermined. In addition, dynamin selectively regulates internalization of P301S tau aggregates, and BIN1/Amphiphysin2 regulates clathrin-mediated endocytosis of P301L tau aggregates (S. Calafate, et al. Cell Rep. 17:931-940, 2016). Given that tau strains found in various tauopathies are markedly different from each other, there is an urgent need to identify the different receptors responsible for the pathologic propagation of toxic tau species such as tau oligomers (F. Clavaguera, et al., Proc. Natl. Acad. Sci. USA, 110:9535-9540, 2013).

### DISCLOSURE

### Technical Problem

However, unlike the research into target receptors corresponding to related beta-amyloid pathways, the investigation into therapeutics for tau-based tauopathies remains an unexplored field.

The present invention is intended to address the above and other problems and aims to provide a pharmaceutical composition for the treatment of tauopathy-associated disorders that can significantly improve cognitive and behavioral impairments by reducing neuronal uptake and propagation of disease-associated tau. However, these tasks are exemplary and the scope of the present invention is not limited thereto.

### Technical Solution

In an aspect of the present invention, there is provided a pharmaceutical composition for treating a tauopathy-related disease, comprising an expression inhibitor or activity inhibitor of RAGE (receptor for advanced glycation end products) as an active ingredient.

In another aspect of the present invention, there is provided a method of treating a subject suffering from a tauopahty-related disease, comprising the step of administering the composition to the subject.

In another aspect of the present invention, there is provided a method of inhibiting propagation of tau protein, comprising the step of treating a neuronal cell or microglial cell with an expression inhibitor or activity inhibitor of RAGE (receptor for advanced glycation end products).

In another aspect of the present invention, there is provided a method for screening therapeutic candidates for the tauopathy-related diseases, comprising the step of treating RAGE (receptor for advanced glycation end products) or cells expressing the RAGE with tau oligomers and at least one test substance; the step of measuring the level of binding between the RAGE and the tau oligomers; and the step of selecting a test substance that significantly reduces the level of binding compared to the control not treated with the test substance.

In another aspect of the present invention, there is provided a method of screening therapeutic candidates for the tauopathy-related diseases, comprising the step of treating the cells expressing RAGE (receptor for advanced glycation end products) with a tauopathy-causing substance selected from the group consisting of i) neurofibrillary tangles (NFTs), ii) pathological brain tissue extracts from a tauopathic patient or a tauopathic model animal, and iii) tau oligomers, and at least one test substance: the step of measuring the level of infection of tau protein into the cells treated with the test substance and the tauopathy-causing substance; and the step of selecting a test substance that significantly reduces the level of infection of the tau protein into the cells compared to the control not treated with the test substance.

### Effect of the Invention

Because the pharmaceutical composition for treating tauopathy-related diseases of the present invention prepared as described above effectively ameliorated tau-induced cognitive and behavioral impairments by reducing neuronal uptake and propagation of disease-associated tau, the present invention can be utilized to develop therapeutic agents that target the neuronal tau propagation process in the early stages of tauopathies. However, the scope of the present invention is not limited by these effects.

### BRIEF DESCRIPTION OF DRAWINGS

FIG. 1a is a schematic representation of the method of cell-based tau infection screening. SH-SY5Y cells were co-transfected with pRFP-N1 and cDNA clones encoding transmembrane proteins, and incubated with 500 nM DyLight 488-tau aggregates for 6 hours. After washing, extracellular fluorescence signals were quenched using trypan blue. RAGE is required for internalization of tau oligomers.

FIG. 1b is a graph showing the results of analyzing relative intracellular DyLight 488 intensities in cells transfected with cDNAs encoding transmembrane proteins. The fluorescent images of putative positive clones were obtained from screen and the average intracellular Dy Light 488 intensities of RFP-positive cells were measured (n=3). pcDNA3 and SDC1 were used as negative and positive controls, respectively. Overexpression of RAGE increases cellular tau infection.

FIG. 1c is a set of graphs showing the results of analyzing cell-bound biotin signal intensities upon treating tau oligomers to WT and Rage KO neurons. The WT and Rage KO neurons were treated with biotin-labeled LMW (left) or HMW (right) tau oligomers for 24 hours, and the cell-bound biotin signal intensities were measured. Rage deficiency reduces tau oligomer binding by ˜40% in primary cultured cortical neurons. The WT and Rage KO neurons were treated with biotin-labeled LMW (left) or HMW (right) tau oligomers for 24 hours, and the cell-bound biotin signal intensities in FIG. 7a were measured. The data were presented as mean±SEM (n=4-6), and the dissociation constants (K_{d}) of binding were obtained from nonlinear regression analysis of saturation binding.

FIG. 1d is a set of microscopic images showing the immunostaining results when WT and Rage KO neurons were treated with tau oligomers. The WT and Rage KO cortical neurons were treated with 500 nM DyLight 488-labeled LMW or HMW tau oligomers for 24 hours. After washing, the cells were immunostained with anti-MAP2b antibodies, and the neuronal tau infection was visualized. RAGE internalizes tau oligomers into neurons. Scale bar, 10 μm.

FIG. 1e is a graph illustrating the quantification of intracellular DyLight 488 signal intensities when WT and Rage KO neurons were treated with tau oligomers. The data are presented as mean±SEM (30 cells per group, n=4), unpaired t-test.

FIG. 1f is a graph showing the results of analyzing intracellular DyLight 488 signal intensities when WT and Rage KO neurons were treated with heparin. The WT and Rage KO neurons were treated with 500 nM DyLight 488-tau oligomers for 24 hours, with or without 15 U/ml heparin. After washing, the cells were immunostained with anti-MAP2b antibodies, and the intracellular DyLight 488 signal intensities in FIG. 7b were measured. RAGE regulates tau infection in an HSPG-independent manner. The data are presented as mean±SEM (30 cells per group, n=3), two-way ANOVA.

FIG. 1g is a set of microscopic images showing immunostaining results after incubation of WT and Rage KO neurons with rTg4510 mouse brain extracts. The WT and Rage KO neurons were incubated for 24 hours with PBS-soluble brain extracts containing 50 ng/ml human tau prepared from a 12-month-old rTg4510 mouse. After washing, the cells were immunostained with anti-human tau (HT7) and anti-MAP2 antibodies (G). RAGE mediates neuronal accumulation of pathology-associated tau. Scale bar, 10 μm.

FIG. 1h is a graph representing the results of quantification of intracellular human tau (HT7) intensities in WT and Rage KO neurons. The data were presented as mean±SEM (30 cells per group, n=3), unpaired t-test.

FIG. 1i is a graph representing the results of quantification of intracellular human tau (HT7) intensities after the treatment of WT and Rage KO neurons with AD CSF. The WT and Rage KO neurons were treated with AD CSF diluted to 1:20 for 24 hours. After washing, the cells were immunostained with anti-human tau (HT7) and anti-MAP2 antibodies, and the intracellular human tau intensities in FIG. 8 were measured. RAGE internalizes tau species present in the CSF of AD patients. The data are presented as mean±SEM (30 cells per group, n=3), unpaired t-test. ***P<0.001, ****P<0.0001.

FIG. 2a is a set of microscopic images of SH-SY5Y cells overexpressing RAGE, after tau treatment. The SH-SY5Y cells overexpressing RAGE were left untreated or treated with 500 nM biotin-labeled tau (biotin-tau) monomers, oligomers, or fibrils for 2 hours. After washing, the cells were incubated with alkaline phosphatase-conjugated streptavidin, followed by BCIP/NBT reaction. RAGE preferentially binds to tau oligomers. Scale bar, 20 μm.

FIG. 2b is a graph showing the results of analyzing cell-bound biotin signal intensities in SH-SY5Y cells overexpressing RAGE after tau treatment. The cell-bound biotin signal intensities were measured and normalized to the signal intensities of untreated cells. The data were presented as mean±SEM (n=3), one-way ANOVA.

FIG. 2c is a graph showing the results of analyzing RAGE binding affinities of tau oligomers (oTau) and Aβ₄₂ oligomers (oAβ₄₂). RAGE-overexpressing SH-SY5Y cells were treated with biotin-tau or Aβ₄₂ oligomers for 2 hours, and the cell-bound biotin signal intensities in FIG. 12a were measured. The data were presented as mean±SEM (n=3-5), and the Kd of binding values were obtained from nonlinear regression analysis of saturated binding.

FIG. 2d is a schematic representation of the constructs comprising mutants that lack full-length (FL) RAGE and extracellular ligand-binding domains (ΔV, ΔC1, and ΔC2), or a cytoplasmic domain (ΔCyto) and functional single nucleotide polymorphisms (G82S). FPS-ZM1 and Azeliragon bind specifically to V domain. SP: signal peptide, V: Ig-like V-type domain, C1: Ig-like C2-type 1 domain, C2: Ig-like C2-type 2 domain, TM: transmembrane domain, Cyto: cytoplasmic domain.

FIG. 2e is a set of microscopic images showing tau infection observed after treating tau oligomers to SH-SY5Y cells transfected with RAGE. The SH-SY5Y cells were transfected with RFP(−) or RFP-tagged FL or mutant (ΔV, ΔC1 and ΔC2) RAGE. The cells were then incubated with 500 nM DyLight 488-tau oligomers for 6 hours, and the cellular tau infection was visualized. RAGE V-C1 domain mediates cellular tau infection. Scale bar, 10 μm.

FIG. 2f is a graph representing the results of quantification of intracellular DyLight 488 signal intensities after treating tau oligomers to SH-SY5Y cells transfected with RAGE. Violin plots representing the median and quartiles (180 cells per group, n=3), one-way ANOVA.

FIG. 2g is a set of microscopic images showing immunostained WT cortical neurons after treating the WT cortical neurons with RAGE antagonists. The WT cortical neurons were treated for 24 hours with 500 nM DyLight 488-tau oligomers in the presence of 1 μM FPS-ZM1 or Azeliragon. After washing, the cells were immunostained with anti-MAP2b antibodies, and the tau infection of neurons was visualized. Blocking the RAGE V domains with antagonists reduces neuronal tau infection. Scale bar, 10 μm.

FIG. 2h is a graph showing the results of quantification of intracellular DyLight 488 signal intensities after treating WT cortical neurons with RAGE antagonists. The data were presented as mean±SEM (40 cells per group, n=3), one-way ANOVA.

FIG. 2i is a set of microscopic images showing SH-SY5Y cells transfected with G82S RAGE and subsequently treated with tau for the observation of tau oligomer binding. The SH-SY5Y cells were transfected with RFP(−) or RFP-tagged WT or G82S RAGE for 24 hours. Then, the cells were either left untreated or treated with 500 nM biotin-tau oligomers for 2 hours. After washing, the cells were incubated with alkaline phosphatase-conjugated streptavidin, followed by BCIP/NBT reaction. RAGE G82S polymorphism enhances binding to tau oligomers. Scale bar, 20 μm.

FIG. 2j is a graph showing the results of analyzing cell-bound biotin signal intensities of SH-SY5Y cells transfected with G82S RAGE and subsequently treated with tau. The signal intensities of transfected cells were normalized to the signal intensities of untreated cells. The data were presented as mean±SEM (n=4, one-way ANOVA. *P<0.05, **P<0.01, ***P<0.001. ****P<0.0001.

FIG. 3a is a schematic diagram illustrating the sketchy structure of a three-chamber microfluidic device for analyzing tau propagation in vitro. Primary hippocampal neurons (DIV 7) of mice were cultured in chambers [WT neurons in the first chamber (C1), and WT or Rage KO neurons in the second and third chambers (C2 and C3)]. The WT neurons in C1 were transduced with a GFP-tau adenoviral vector for tau oligomer formation.

FIG. 3b is a set of gel images representing the results of dot blot analysis, showing whether detergent-insoluble tau aggregates were generated in the primary hippocampal neurons infected with GFP-tau adenovirus. Different groups of WT neurons were left untreated, infected with GFP-tau adenovirus (GFP-Tau AdV, MOI 50), or treated with 500 nM tau oligomers for 24 hours. After 48 hours, the formation of intracellular tau aggregates was assessed by dot blot analysis.

FIG. 3c is a set of fluorescence microscopic images showing tau propagation in neurons after GFP-Tau infection. Fourteen days after GFP-Tau transfection in C1 neurons, propagation of GFP-tau protein was detected, from C1 to C2 and C3 neurons. Rage deficiency reduces synaptic tau propagation in vitro. Scale bar, 20 μm.

FIG. 3d is a graph showing the results of quantification of GFP signal intensities in WT and Rage KO neurons. The data are presented as mean±SEM (n=3), unpaired t-test.

FIG. 3e is a schematic diagram of the experimental method and timeline for injecting AD-tau into experimental animals (mice). The red dots indicate the injection sites.

FIG. 3f is a set of microscopic images of ipsilateral hippocampi of WT or RAGE KO mice after AD-tau (8 g/mouse) injection, along with a graph showing the results of analyzing AT8 signal intensities. Pathologic tau propagation was reduced in the RAGE KO mouse brain.

FIG. 3g is a set of microscopic images of ipsilateral cortices of WT and RAGE KO mice after AD-tau (8 g/mouse) injection, along with a graph showing the results of analyzing AT8 signal intensities. Scale bar, 100 μm. The data are presented as mean±SEM (n=5 per group), two-way ANOVA.

FIG. 3h shows the distribution of AD-tau after AD-tau injection in WT or RAGE KO mice. The distributions of AT8-positive tau pathology (red dots) were observed in coronal sections (Bregma 0.86, −1.82, −3.08 and −4.48 mm) of the brains at 6 months after the injection. *P<0.05, **P<0.005, ***P<0.001.

FIG. 4a is a set of immunohistochemical microscopic images depicting the level of neuronal RAGE expression in the CAI region of the hippocampus of an rTg4510 mouse. Hippocampal brain sections from 12-month-old NonTg mice and age-matched rTg4510 mice were immunostained with anti-RAGE, anti-phospho-tau202/205 (AT8), and anti-MAP2 antibodies. RAGE is required for tau-based behavioral deficits. Scale bar, 10 μm.

FIG. 4b is a graph representing the results of quantification of neuronal RAGE signal intensities in the CAI region of the hippocampus of an rTg4510 mouse. The data were presented as mean±SEM (20 cells per group, n=3), unpaired t-test.

FIG. 4c is a set of gel images showing the results of immunoblotting analysis of the levels of RAGE expression and intercellular human tau in WT and RAGE KO cortical neurons. The WT and Rage KO cortical neurons were treated with 100 nM tau oligomers for 48 hours. Extracellular tau oligomers increase RAGE expression in neurons. The data were presented as mean±SEM (n=3), two-way ANOVA.

FIG. 4d is a set of graphs showing quantification of the RAGE and HT7 expression levels in WT and Rage KO cortical neurons.

FIG. 4e is a set of graphs showing the results of the Y-maze tests after injection of tau adeno-associated viruses in WT and Rage KO mice. The GFP-P301L tau adeno-associated viruses were injected intracranially into the left hippocampal region of 3-month-old WT and Rage KO mice. RAGE deficiency delays cognitive impairment induced by unilateral viral expression of GFP-Tau in hippocampus. The data were presented as means±SEM (n=4 per group), unpaired t-test.

FIG. 4f is a graph showing the results of novel object recognition tests performed after injection of tau adeno-associated viruses in WT and Rage KO mice. The data are presented as mean±SEM (n=3 per group), unpaired t-test.

FIG. 4g is a graph showing the results of passive avoidance tests performed after injection of tau adeno-associated viruses in WT and Rage KO mice. The data are presented mean±SEM (n=4 per group), unpaired t-test.

FIG. 4h is a set of graphs representing the results of the Y-maze tests performed after administration of RAGE antagonists in WT and Rage KO mice. Littermates of two-month-old NonTg mice and rTg4510 mice were injected intraperitoneally with vehicle (5% DMSO) or FPS-ZM1 (1 mg/kg/day) daily for 2.5 months, respectively. Administration of RAGE antagonists ameliorated the behavioral impairment of the rTg4510 mice. The data were presented as mean±SEM (n=6-13 per group), one-way ANOVA.

FIG. 4i is a graph representing the results of novel object recognition test performed after administration of RAGE antagonists in WT and Rage KO mice. The data were presented as mean±SEM (n=8-15 per group), one-way ANOVA.

FIG. 4j is a graph representing the results of passive avoidance test performed after administration of RAGE antagonists in WT and Rage KO mice. The data were presented as mean±SEM (n=9-14 per group), paired t-test. NS, not significant,*P<0.05, **P<0.01, ***P<0.001, ****P<0.0001.

FIG. 5a, which relates to the preparation of tau protein, is a graph representing the results of size exclusion chromatography analysis of the reaction products using a Superose 6 column. The purified tau protein was incubated after stirring with heparin for 24 hours at 37° C. The absorbance at 280 nm of each fraction was monitored. n represents the estimated number of tau monomers corresponding to the peak.

FIG. 5b, which relates to the preparation of tau protein, is a graph showing the results of size exclusion chromatography analysis of the reaction products using a Superdex 200 column. The purified tau protein was incubated with heparin at 37° C. for 1 hour (red) or 1.5 hours (blue) without stirring at room temperature.

FIG. 5c, which relates to the preparation of tau protein, is a photograph showing the monomeric, oligomeric, and fibrillary forms of tau protein subjected to native PAGE and stained with Coomassie Brilliant Blue.

FIG. 6a is a graph showing the analysis of relative intracellular DyLight 488 signal intensities after transfection of SH-SY5Y cells as the primary outcome of cell-based tau infection screening. The SH-SY5Y cells were co-transfected with pRFP-N1 and cDNA clones encoding transmembrane proteins, and incubated with 500 nM DyLight 488-tau aggregates for 6 hours. After washing, the extracellular fluorescence signal was quenched with trypan blue. Fluorescence images were acquired, and the average intracellular DyLight 488 intensity of RFP-positive cells was measured.

FIG. 6b is a set of fluorescence microscopic images showing relative intracellular DyLight 488 signals after transfection of SH-SY5Y cells as the primary outcome of cell-based tau infection screening. pcDNA3 (Control) and SDC1 were used as negative and positive controls, respectively. Scale bar, 20 μm.

FIG. 7a is a set of microscopic images of WT and Rage KO neurons after treatment with biotin-labeled LMW or HMW tau oligomers. The WT and Rage KO neurons were treated with biotin-labeled LMW or HMW tau oligomers for 24 hours. After washing, the cells were incubated with alkaline phosphatase-conjugated streptavidin followed by BCIP/NBT reactions. RAGE deficiency in primary cortical neurons reduced cellular binding and internalization of tau oligomers.

FIG. 7b is a set of fluorescence microscopic images showing tau infection in WT and Rage KO neurons treated with heparin and DyLight 488-labeled LMW or HMW tau oligomers. The WT and Rage KO neurons were treated with 500 nM DyLight 488-labeled tau oligomers for 24 hours regardless of the presence or absence of 15 U/ml heparin. After washing, the cells were immunostained with anti-MAP2b antibodies, and the tau infection in neurons was visualized. Scale bar, 10 μm.

FIG. 8 is a set of fluorescence microscopic images showing tau infection in WT and Rage KO neurons treated with AD CSF and subsequently immunostained with antibodies. The WT and Rage KO neurons were treated with AD CSF diluted to 1:20 for 24 hours. After washing, the cells were immunostained with anti-human tau (HT7) and anti-MAP2 antibodies. Tau species present in the CSF of AD patients enter neurons via RAGE. Scale bar, 10 μm.

FIG. 9a is a set of fluorescence microscopic images showing tau infection after injection of rTg4510 mouse brain extracts into WT and Rage KO mice. PBS-soluble brain extracts prepared from a 12-month-old rTg4510 mouse (1 μg/ml human tau) was injected into the prefrontal cortices of three-month-old WT and Rage KO mice. 48 hours later, brain sections from the prefrontal cortices were immunostained with anti-human tau (HT7) and anti-MAP2 antibodies. Rage deficiency blocks neuronal tau infection in vivo. Scale bar, 10 μm.

FIG. 9b is a graph showing the results of percentage analysis of human tau-positive neurons and glial cells after injection of rTg4510 mouse brain extracts into WT and Rage KO mice. The data were presented as mean±SEM (n=3 per group). NS, not significant: *P<0.05, unpaired t-test.

FIG. 10a is a graph showing the results of analyzing DyLight 488 signal intensities of microglia from WT and Rage KO mice. The WT and Rage KO microglia were treated with 100 nM DyLight 488-tau oligomers for 24 hours, and after washing, the cells were immunostained with anti-Iba-1 antibodies. Cellular tau infection was visualized and intracellular DyLight 488 signal intensities were measured. Scale bar, 10 μm. The data were presented as mean±SEM (50 cells per group, n=4). NS, not significant, ****P<0.0001, unpaired t-test.

FIG. 10b is a graph showing the results of analyzing DyLight 488 signal intensities in astrocytes from WT and Rage KO mice. The WT and Rage KO astrocytes were treated with 100 nM DyLight 488-tau oligomers for 24 hours and immunostained with anti-GFAP antibodies.

FIG. 11a is a set of immunoblotting gel images showing the interaction between RAGE and His-tau protein. HEK293T cell lysates were prepared and incubated with monomeric, oligomeric, or fibrillar forms of tau. The interaction between RAGE and His-tau protein was evaluated in a pull-down assay using Ni-NTA and immunoblotting. RAGE preferentially bound to tau oligomers.

FIG. 11b is a graph for observation of the interaction between RAGE and His-tau protein, showing the results of quantification of His-tau-bound RAGE levels. The data were presented as mean±SEM (n=3), one-way ANOVA.

FIG. 11c is a set of immunoblotting gel images for observation of the interaction between overexpressed RAGE-GFP and tau protein. The interaction between overexpressed RAGE-GFP and tau protein was evaluated in a co-immunoprecipitation (IP) assay using anti-GFP antibodies and immunoblotting.

FIG. 11d is a graph showing the results of quantification of RAGE-GFP-bound tau levels. The data were presented as mean±SEM (n=3), one-way ANOVA. *P<0.05, **P<0.01.

FIG. 12a is a set of microscopic images for comparison of the binding and uptake of tau and Aβ₄₂ oligomers in RAGE-overexpressing SH-SY5Y cells. The SH-SY5Y cells were treated with biotin-labeled tau or Aβ₄₂ oligomers for 2 hours. After washing, cells were incubated with alkaline phosphatase-conjugated streptavidin followed by BCIP/NBT reaction. Scale bar, 20 μm.

FIG. 12b is a graph showing the results of analyzing DyLight 488 and FITC intensities in RAGE-overexpressing SH-SY5Y cells. The cells were treated with increasing concentrations of DyLight 594-tau oligomers (red) in the presence of 125 nM FITC-Aβ₄₂ oligomers (green) for 2 hours. Fluorescence images were acquired, and intracellular DyLight 594 and FITC intensities were measured. The data were presented as mean±SEM (n=3).

FIG. 12c is a set of graphs showing the results of analyzing DyLight 488 and FITC intensities in RAGE-overexpressing SH-SY5Y cells after treatment with FPS-ZM1 or Azeliragon. The cells were treated with increasing concentrations of FPS-ZM1 (left) or Azeliragon (right) for 4 hours in the presence of 500 nM DyLight 488-tau or 125 nM FITC-Aβ₄₂ oligomers. Fluorescence images were acquired, and intracellular DyLight 488 and FITC intensities were measured. The data were presented as mean±SEM (n=4).

FIG. 13a is a set of fluorescence microscopic images for observation of tau infection in SH-SY5Y cells, showing the mapping of RAGE domains responsible for tau binding and uptake. The SH-SY5Y cells were transfected with RFP(−) or RFP-tagged RAGE full-length (FL) or cytoplasmic domain deletion mutants (ΔCyto). The cells were then incubated with 500 nM DyLight 488-tau oligomers for 6 hours, and cellular tau infection was visualized. Scale bar, 10 μm.

FIG. 13b is a graph showing the results of quantification of DyLight 488 signal intensities in SH-SY5Y cells. Violin plots showing median and quartiles (100 cells per group, n=3), one-way ANOVA.

FIG. 13c is a set of immunoblotting gel images for observation of the expression after transfection of SH-SY5Y cells with WT or G82S RAGE-FLAG. The SH-SY5Y cells maintained in culture medium containing high or low glucose were transfected with WT or G82S RAGE-FLAG and treated with 0.5 μg/ml tunicamycin for 24 hours. The molecular mass of WT and G82S RAGE was analyzed by immunoblotting. The data were presented as mean±SEM (n=3), unpaired t-test. *P<0.05, ****P<0.0001.

FIG. 13d is a graph showing the results of analyzing relative RAGE-FLAG levels after transfection of SH-SY5Y cells with WT or G82S RAGE-FLAG. The ratio of glycosylated and non-glycosylated forms of RAGE-FLAG was measured.

FIG. 14a is a set of immunoblotting gel images showing the results of analyzing cytoplasmic RAGE expression and nuclear NF-κB p65 levels after treatment of SH-SY5Y cells with tau oligomers. The data were presented as mean±SEM (n=3), one-way ANOVA.

FIG. 14b is a set of graphs showing the results of quantification of cytoplasmic RAGE (left) and nuclear NF-κB p65 (right) levels after treatment of SH-SY5Y cells with tau oligomers.

FIG. 14c is a set of immunoblotting gel images showing the results of analyzing cytoplasmic RAGE expression and nuclear NF-κB p65 levels after treatment of SH-SY5Y cells with tau oligomers in the presence of FPS-ZM1 or Azeliragon. The SH-SY5Y cells were treated with tau oligomers in the presence of 1 μM FPS-ZM1 or Azeliragon for 48 hours. The data were presented as mean±SEM (n=4), one-way ANOVA. *P<0.05, **P<0.01, ***P<0.001, ****P<0.0001.

FIG. 14d is a set of graphs showing the results of quantification of cytoplasmic RAGE (left) and nuclear NF-κB p65 (right) levels after treatment of SH-SY5Y cells with tau oligomers.

FIG. 15a is a schematic diagram depicting the analysis procedure for tau propagation in vivo in WT and Rage KO mice. GFP-P301L tau adeno-associated viruses (GFP-Tau AAV, 6.5×10¹⁰ ifu/ml, 5 μl) were injected intracranially into the left hippocampi of 3-month-old WT and Rage KO mice. Rage KO impairs tau oligomer propagation after GFP-Tau expression in hippocampus.

FIG. 15b is a set of fluorescence microscopic images for observation of tau propagation in vivo in WT and Rage KO mice. RAGE deficiency reduces neuronal tau propagation by ˜20% in vivo. Five months after GFP-Tau AAV injection, hippocampal brain sections were immunostained with anti-human oligomeric tau (T22) antibodies. Scale bar, 500 μm.

FIG. 15c is a graph showing the results of analyzing GFP signal intensities in the ipsilateral hippocampi of WT and Rage KO mice.

FIG. 15d is a graph showing the results of analyzing T22 signal intensities in the contralateral hippocampal CA3 regions of WT and Rage KO mice. Bars were presented as mean±SEM (n=4 per group), unpaired t-test. NS, not significant, *P<0.05.

FIG. 16a is a set of graphs representing the results of the Y-maze test performed after administration of RAGE antagonists in experimental mice, two-month-old tTA-negative mice and rTg4510 littermates were injected intraperitoneally with vehicle (5% DMSO) or Azeliragon (1 mg/kg/day) daily for 2.5 months. Administration of RAGE antagonists ameliorates behavioral impairment in rTg4510 mice. The data were presented as mean±SEM (n=6-11 per group), one-way ANOVA. NS, not significant, *P<0.05, **P<0.01.

FIG. 16b is a graph representing the results of novel object recognition test performed after administration of RAGE antagonists in experimental mice. The data were presented as mean±SEM (n=5-8 per group) one-way ANOVA.

FIG. 16c is a graph representing the results of passive avoidance test performed after administration of RAGE antagonists in experimental mice. The data were presented as mean±SEM (n=8-10 per group), paired t-test.

FIG. 17 is a schematic diagram representation illustrating the RAGE-mediated pathogenesis in Alzheimer's disease, which is a tauopathy-related disorder. RAGE has previously been reported to bind to Aβ oligomers (oAβ) and induce neurotoxicity. The present inventors identified that RAGE acts on the neuronal uptake and propagation of tau oligomers (oTau). It has been observed that RAGE also affects the uptake of tau oligomers in microglial cells: thereby, it is expected that RAGE plays a role in the inflammatory response of the microglial cells. In addition, since RAGE is a receptor that functions to allow substances in the blood vessels to pass into the brain through the blood-brain barrier, it is expected to act on the passage of tau oligomers across the blood brain barrier and cause tauopathy in the brain. Therefore, antagonistic small molecule drugs or anti-RAGE antibodies targeting the V-C1 domain of RAGE, such as FPS-ZM1 or Azeliragon, can be used to inhibit tauopathy progression by disturbing protein-protein interactions between tau oligomers and RAGE.

FIG. 18a is a set of microscopic images showing the immunostaining results after treatment of WT hippocampal neurons with anti-RAGE antibodies that bind to RAGE V domains. Blocking the RAGE V domains using anti-RAGE antibodies reduces neuronal tau infection. Scale bar, 10 μm.

FIG. 18b is a graph showing the results of quantification of intracellular DyLight 488 signal intensities after treatment of WT hippocampal neurons with anti-RAGE antibodies. The data were presented as mean±SEM (17-23 cells per group), unpaired t-test, **P<0.01.

FIG. 19a is a set of microscopic images for observation of tau propagation between cells using a tau-BiFC (bimolecular fluorescence complementation) system. Tau propagation increases when VN-tau-expressing cells and tau-VC-expressing cells are transfected with RAGE and co-cultured, and decreases when they are co-cultured with anti-RAGE antibodies.

FIG. 19b is a graph showing the results of quantification of fluorescence intensities upon transfection of VN-tau-expressing cells and tau-VC-expressing cells with RAGE followed by co-culture with anti-RAGE antibodies in a tau-BiFC system. The data were presented as median and interquartiles and min-max (200 cells per group), one-way ANOVA, ****P<0.0001.

### DETAILED DESCRIPTION

### Definitions of Terms

As used herein, the term “tau” refers to a microtubule protein found inside brain nerve cells that plays an important role in axonal transport and neuronal integrity, and whose misfolding is known to destroy nerve cells and cause dementia. Normally, tau proteins fold into a specific shape, but when abnormal tau proteins fold, they take on a different shape. The abnormal tau molecules get extra phosphate groups, which affects the protein's arrangement. With this altered structure, the tau protein exhibits different activities within nerve cells, becoming hazardous as it tangles together into clumps in dendrites and blocks the transmission of electrical impulses.

As used herein, the term “tau oligomer”, also referred to as “tau aggregate”, is an insoluble tau protein formed when tau protein is hyperphosphorlyated for any reason, and its formation is thought to be closely related to pathogenesis of tauopathies.

As used herein, the term “tau infection” refers to the process of intracellular delivery of pathogenic tau proteins, such as tau oligomers. Although tauopathic-pathogenic proteins such as tau oligomers are not infectious organisms such as viruses or bacteria, the term “infection” is used to describe the process of intracellular delivery of tau proteins, since they are absorbed by neuronal-associated cells like target neurons or microglia, and spread through trans-synaptic transmission to other neurons in a manner similar to infectious agents such as viruses or bacteria.

As used herein, the term “tauopathy” refers to a neurodegenerative brain disorder associated with dementia, which is caused by the accumulation of abnormal tau protein in the brain. Tauopathy belongs to a group of neurodegenerative diseases associated with the aggregation of tau protein into neurofibrillary or fibrillary tangles (NFTs) in the human brain. The tangles are formed by hyperphosphorylation of microtubule protein known as tau, which causes the protein to dissociate from microtubules and form insoluble aggregates.

As used herein, the term “RAGE (receptor for advanced glycation end products)” refers to an end-glycation product receptor, which promotes neuronal infection and propagation of pathogenic tau and mediates behavioral abnormalities. The present invention identifies a role for RAGE in reducing neuronal infection and propagation of disease-associated tau in the early stages of tauopathy.

### DETAILED DESCRIPTIONS OF THE INVENTION

In an aspect of the present invention, there is provided a pharmaceutical composition for treating a tauopathy-related disease, comprising an expression inhibitor or activity inhibitor of RAGE (receptor for advanced glycation end products) as an active ingredient.

In the pharmaceutical composition, the activity inhibitor of RAGE may be capable of binding specifically to the RAGE V domain, and the tauopathy-related disease may be selected from a group comprising Alzheimer's disease, Parkinson's disease, corticobasal degeneration, dementia, chronic traumatic encephalopathy, progressive supranuclear palsy, corticobasal degeneration, Ganglioglioma, Gangliocytoma, Meningioangiomatosis, Subacute sclerosing panencephalitis, Encephalopathy, tuberous sclerosis, pantothenate kinase-associated neurodegeneration, and lipofuscinosis. The dementia may be vascular dementia, plaque-free primary age-related tauopathy dementia, or frontotemporal dementia.

In the pharmaceutical composition, the expression inhibitor may be an shRNA or an antisense nucleic acid, and the activity inhibitor may be an antibody that specifically binds to RAGE, an antigen-binding fragment thereof, a RAGE antagonizing peptide (RAP), FPS ZM1, or Azeliragon. The RAGE antagonizing peptide may comprise the amino acid sequence of SEQ ID NO: 17 (ELKVLMEKEL).

In another aspect of the present invention, there is provided a method of treating a tauopathy-related disease, comprising the step of administering the composition to a subject suffering from tauopathy-related disease.

In another aspect of the present invention, there is provided a method of inhibiting propagation of tau protein, comprising the step of treating a neuronal cell or microglia with an expression inhibitor or activity inhibitor of RAGE (receptor for advanced glycation end products).

In another aspect of the present invention, there is provided a method for screening therapeutic candidates for treating tauopathy-related diseases, comprising the step of treating RAGE (receptor for advanced glycation end products) or cells expressing the RAGE with tau oligomers and at least one test substance: the step of measuring the level of binding between the RAGE and the tau oligomers; and the step of selecting a test substance that significantly reduces the level of binding compared to the control not treated with the test substance.

In the method of screening, the tau oligomers may be fluorescently labeled, and the level of binding may be measured using methods such as surface plasmon resonance (SPR), yeast two-hybrid assay, biolayer interferometry (BLI), immunoprecipitation (IP), or radioimmunoassay (RIA).

In another aspect of the present invention, there is provided a method of screening therapeutic candidates for the tauopathy-related diseases, comprising the step of treating the cells expressing RAGE (receptor for advanced glycation end products) with a tauopathy-causing substance selected from the group consisting of i) neurofibrillary tangles (NFTs), ii) pathological brain tissue extracts from a tauopathic patient or a tauopathic model animal, and iii) tau oligomers, and at least one test substance: the step of measuring the level of infection of tau protein into the cells treated with the test substance and the tauopathy-causing substance; and the step of selecting a test substance that significantly reduces the level of infection of the tau protein into the cells compared to the control not treated with the test substance.

In the screening method described above, the test compound may be a substance that has been preliminarily screened by a candidate screening method under in vitro conditions comprising the steps of treating RAGE (receptor for advanced glycation end products) or cells expressing the RAGE with tau oligomers and at least one test substance: the step of measuring the level of binding between the RAGE and the tau oligomers; and the step of selecting a test substance that significantly reduces the level of binding compared to the control not treated with the test substance.

In the method of screening, the test substance may be a small compound, an extract of a microorganism, plant or animal, an antibody specific binding to RAGE or tau protein, and siRNA, shRNA or antisense nucleotide that inhibits the expression of the RAGE.

In the method of screening, the tau oligomers may be fluorescently labeled, and the ‘cells’ may be neurons or microglia.

The pharmaceutical composition according to one embodiment of the present invention may comprise a pharmaceutically acceptable carrier, and may further comprise a pharmaceutically acceptable adjuvant, excipient, or diluent apart from the carrier.

As used herein, the term “pharmaceutically effective amount” means an amount sufficient to inhibit or mitigate increased vascular permeability with a reasonable benefit/risk ratio applicable to the medical use, and the effective dose level may be determined based on factors including the individual's type, severity, age, gender, activity of the drug, sensitivity to the drug, time of administration, route of administration and rate of elimination, duration of treatment, concomitant medications, and other factors well known in the medical field. The composition of the present invention may be administered as an individual therapeutic agent or in combination with other therapeutic agents, and may be administered sequentially or simultaneously with conventional therapeutic agents. They can be administered singly or in multiple doses. It is important to take into account all of the above factors, and administer an amount that will provide maximum benefit in a minimal amount without side effects, which can be readily determined by those skilled in the art.

Examples of the carriers, excipients and diluents mentioned above, are lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oil. Fillers, anti-flocculants, lubricants, wetting agents, flavors, emulsifiers, preservatives, etc. can be further included.

In addition, the pharmaceutical composition according to one embodiment of the present invention may be formulated using methods known in the art, to allow rapid release, or, sustained or delayed release of the active ingredient upon administration to a mammal. The formulations include powders, granules, tablets, emulsions, syrups, aerosols, soft or hard gelatin capsules, sterile injectable solutions, and sterile powder forms.

The pharmaceutical composition according to one embodiment of the present invention may be administered by various routes, for example, orally, parenterally, by suppository, transdermally, intravenously, intra-abdominally, intra-peritoneally, intramuscularly, intralesionally, intranasally, intrathecally, or intrathecally, and can also be administered using an implantable device for sustained or continuous or repeated release. The number of doses may be administered once or in multiple doses a day, within any desired range, and the duration of administration is not particularly limited. Furthermore, the pharmaceutical composition of the present invention may be administered at a dose of 0.1 mg/kg to 1 g/kg, and more preferably at a dose of 1 mg/kg to 600 mg/kg. Meanwhile, the dosage may be appropriately adjusted according to the age, gender and condition of the patient.

Tauopathy is a neurodegenerative disease caused by the hyperphosphorylation and aggregation of tau protein abnormally accumulated in nerve cells, which has been implicated as a cause of several neurodegenerative brain diseases. The aggregates of tau protein seen in patients with tau disease are primarily found in the cell bodies and dendrites of nerve cells, called neurofibrillary tangles (NFTs) and neuropil threads. In neurofibrillary tangles, tau proteins are composed of paired helical filaments (PHFs) that are tangled into fine threads, which are aggregated and hyperphosphorylated unlike normal tau proteins. Although it is not known exactly what role the abnormal aggregation of tau proteins in tauopathies plays in the advanced stages of the disease, it is similar to the aggregation phenomenon common in neurodegenerative diseases.

In tauopathies, misfolded tau proteins exhibit synaptic transmission propagation between neurons. However, the underlying mechanism by which the tau proteins enter neurons during pathologic propagation is unclear. The present invention identifies that RAGE (receptor for advanced glycation end products) promotes neuronal uptake and propagation of pathogenic tau and mediates behavioral abnormalities.

The present inventors selectively stimulated the endocytosis of tau oligomers by isolating RAGE from a cell-based functional screening of the entire genome for 1,523 complementary DNAs which encode transmembrane proteins. RAGE deficiency reduced neuronal uptake and propagation of disease-associated tau in vitro and in vivo. The RAGE was upregulated in the brain of rTg4510 mice, and the treatment with FPS-ZM1 or Azeliragon, which are RAGE-specific antagonists, significantly alleviated cognitive impairment. These results suggest that neuronal RAGE plays an important role in promoting synaptic tauopathy progression and tau-mediated memory impairment.

### MODE FOR CARRYING OUT THE INVENTION

The present invention will now be described in more detail with reference to the following examples. However, the invention is not limited to the embodiments disclosed herein, but could be embodied in many different forms, and the following embodiments are provided to make the disclosure of the invention complete and to give those of ordinary skill in the art a complete idea of the scope of the invention.

### Example 1: Cell-Based Screening of Tau Infection-Related Receptors

The present inventors performed a cell-based tau transfection receptor screening using a cDNA expression library. First, SH-SY5Y cells were transfected with pRFP-N1 and mammalian expression vectors comprising cDNA encoding each human and mouse transmembrane protein (1,523 in total) for 24 hours. pcDNA3 and SDC1 cDNAs were used as negative and positive controls, respectively. The cells were then treated with 500 nM DyLight 488-tau aggregates for 6 hours, washed with PBS, and the extracellular DyLight 488 signals were quenched with 0.05% trypan blue (Sigma-Aldrich). The intracellular infection of the above tau aggregates was then visualized using INCell Analyzer 2000 (GE Healthcare), and the intensities of intracellular DyLight 488 signal in RFP-positive cells were measured using Image J.

### Example 2: Analyzing Tau Infection

The present inventors performed a tau infection assay in primary cultured cells. First, primary cortical neurons or hippocampal neurons (DIV 7) isolated from WT and Rage KO mice were incubated with 500 nM DyLight 488-tau oligomers for 24 hours. HSPG-mediated tau internalization was blocked by co-treatment with 15 U/ml heparin (Sigma-Aldrich). Subsequently, the neurons were treated with 1 μM of two RAGE antagonists, FPS-ZM1 (Calbiochem) and Azeliragon (MedChemExpress), respectively of for evaluation. Or, the neurons were treated with 1 μg/ml of anti-RAGE antibodies (Invitrogen, PA5-78736) for evaluation. To investigate intracellular infection of pathogenic tau, the neurons were incubated for 24 hours with PBS-soluble rTg4510 brain extracts containing 50 ng/ml human tau or CSF diluted to 1:20 prepared from human Alzheimer's disease patients. Then, primary cortical microglia and astrocytes from the WT and Rage KO mice (DIV 14) were incubated with 100 nM DyLight 488-tau oligomers for 24 hours. The cells were then washed with PBS, fixed with 4% paraformaldehyde (Sigma-Aldrich), and immunocytochemistry was performed. Images were acquired using a confocal laser scanning microscope LSM700 (Carl Zeiss), and the intracellular tau signal intensities were measured using Image J.

### Example 3: Recombinant Tau Purification, Fluorescent Labeling, and Fibrosis

The human 0N4R tau of the present invention was subcloned into a pET-His vector with reference to the method described in a prior art (Y. Kim, et al., Neurobiol. Dis. 87:19-28, 2016) and the 6×His tagged human 0N4R tau was expressed in bacteria (BL21-DE3) and purified using Ni-NTA agarose (Qiagen). The purified tau monomers were incubated with DyLight 488 or 594 NHS Ester (Thermo Scientific) for 1 hour at room temperature for fluorescent labeling. Subsequently, 24 μM tau monomers were incubated with 5 mM dithiothreitol (GoldBio) and 6 μM heparin in PBS to induce fibrosis. For subsequent tau oligomerization, the mixture was incubated for 1 h (low molecular weight) or 1.5 h (high molecular weight) at room temperature without agitation. Tau fibrils were prepared by incubating the mixture at 37° C. for 24 hours with constant stirring at 1,000 rpm, and the molecular sizes of the tau oligomers and fibrils were determined by fast protein liquid chromatography (FPLC). That is, the tau protein was filtered through a 0.2 μm membrane and separated through a Superose 6 or Superdex 200 incremental 10/300GL column (GE Healthcare). The fractions were then collected and the presence of tau protein was monitored by absorbance at 280 nm. Tau protein was also subjected to native PAGE and stained with Coomassie Brilliant Blue (USB) to determine its molecular size.

### Example 4: Preparation of Aβ₄₂ Oligomers

For the preparation of the oligomers, the present inventors dissolved the synthetic biotin-Aβ₄₂ peptide (rPeptide) at 2 mM in DMSO and diluted it in PBS to obtain a 100 M stock solution. The reactant was then incubated at 22° C. for 16 hr and then the supernatant was collected after centrifuging at 16,000×g for 15 min. FITC-Aβ₄₂ oligomers were prepared according to the previously reported method (T.-I. Kam, et al., Clin. Invest. 123:2791-2802, 2013). FITC-Aβ₄₂ peptides (rPeptides) were dissolved in DMSO at 2 mM and diluted to a final 125 M stock solution in PBS. It was then incubated at 4° C. for 24 hrs, centrifuged at 12,000×g for 10 min, the supernatant was collected and stored at −80° C. until use.

### Example 5: Experimental Animals

The rTg4510 mice used in the present invention were obtained by crossing a human P301L tau responder strain (The Jackson Laboratory, #015815) to a tetracycline-regulated transactivator (tTA) strain (The Jackson Laboratory, #016198). Mice not carrying the CaMKII-TA transgene were used as a control and all mice used in the present invention were maintained in a pathogen-free specific animal facility. All experiments were performed in accordance with the guidelines for animal research of the Ministry of Food and Drug Safety (MFDS) and the protocols were approved by the Institutional Animal Care and Use Committee (IACUC) of Seoul National University. In addition, RAGE knockout (KO) mice on a C57BL/6 background were provided by Dr. Ann Marie Schmidt (New York University School of Medicine) and Dr. Stefanie Vogel (University of Maryland School of Medicine), and littermates of WT and RAGE knockout mice were used in the experiments. All rearing and procedures were performed in accordance with the NIH Guide for the Care and Use of Laboratory Animals and were approved by the Johns Hopkins University Animal Care and Use Committee.

### Example 6: Manufacturing the Rage KO Mice

The present inventors generated Rage-deficient mice from embryos obtained from the European Mouse Mutant Archive (EMMA) (EM ID: 02352, LEXKO-2071). The embryos provided by EMMA already had a deletion between exons 2 and 4 of the Rage gene, leaving only one LoxP site. The sequence corresponding to the LoxP cleavage site was verified by direct sequencing (Bionics Co., Ltd., Seoul, Korea). Genotyping of the target allele was performed by PCR analysis using primers (SEQ ID NOs: 13 and 14). The above mice were backcrossed and maintained on a C57BL/6N background.

### Example 7: Preparation of rTo4510 Brain Extract

For the preparation of rTg4510 brain extracts, the present inventors anesthetized 12-month-old rTg4510 mice and perfused them with PBS containing 10 U/ml heparin. The brains of the mice were then excised, frozen in liquid nitrogen, and homogenized in 5 volumes (wt/vol) of PBS. The homogenate was then centrifuged at 3,000×g for 5 min at 4° C. and the supernatant was collected, and the concentration of human tau was determined using a human tau (total) ELISA kit (Invitrogen) according to the manufacturer's instructions.

### Example 8: Collect Cerebrospinal Fluid (CSF) from AD Patients

The present inventors collected cerebrospinal fluid (CSF) from human Alzheimer's disease patients. Specifically, human CSF was obtained from a routine lumbar puncture between L3/L4 or L4/L5 between 8:00 am and 12:00 pm. Within 4 hours of the lumbar puncture, CSF was centrifuged at 2,000×g for 10 min and the supernatant was aliquoted into 1 ml polypropylene vials and stored at −80° C. until use. The levels of CSF Aβ₄₂, total tau, and phospho-tau181 (triple marker) were measured using the INNOTEST β-AMYLOID₍₁₋₄₂₎, hTAU Ag, and PHOSPHO-TAU_{(181P)} ELISA kits (Fujirebio Europe, Gent, Belgium) according to the manufacturer's instructions. The invention was approved by the ethics committees of Seoul National University Bundang Hospital and Seoul National University, and all participants consented to the use of their clinical data for research purposes.

### Example 9: Cell Culture and DNA Transduction

The SH-SY5Y cells, SH-SY5Y cells expressing VN-tau, SH-SY5Y cells expressing tau-VC, and HEK293T cells of the present invention were maintained in DMEM/high glucose medium (HyClone) containing 10% fetal bovine serum (FBS, Gibco), 100 U/ml penicillin-streptomycin (Gibco), and 10 μ/ml gentamicin (Gibco), and subsequently incubated in 5% CO₂ at 37° C. in the atmosphere and transfected with Lipofector-pMAX (AptaBio) or polyethylenimine (Sigma-Aldrich) according to the manufacturer's instructions. When necessary, the cells were maintained in DMEM/low glucose medium (HyClone) and treated with tunicamycin (Sigma-Aldrich). When utilizing a tau-BiFC system, the VN-tau expressing SH-SY5Y cells and tau-VC expressing SH-SY5Y cells were cocultured in symbiosis.

### Example 10: Construction of Plasmids

For plasmid construction, a gene encoding human RAGE was amplified by PCR from a cDNA library and subcloned into pEGFP-N1, 3×FLAG-CMV-14, or pRFP-N1. VN deletions (ΔV, ΔC1, ΔC2, and Δinto) and G82S RAGE mutant and P301L tau mutant were generated by site-directed mutagenesis. All cDNA constructs were confirmed by DNA sequencing analysis. The restriction enzyme sites used for digestion and the plasmid information and primer sequences used for generation of the mutants are summarized in Tables 1 and 2 below.

TABLE 1
Information of plasmids

| | | Inserted | | Cloning | |
|---|---|---|---|---|---|
| Plasmids | Tags | cDNA | Vectors | Sites | |
| His-human | His | 0N4R tau | pET-His | BamHI | Xhol |
| 0N4R tau | | | | | |
| (bacteria) | | | | | |
| GFP-human | GFP | 0N4R tau | pEGFP-C1 | EcoRI | BamHI |
| 0N4R tau | | | | | |
| GFP-human | N/A | GFP-0N4R | pJDK | EcoRV | Xbal |
| 0N4R P301L | | P301L tau | | | |
| tau(AAV) | | | | | |
| RAGE-GFP | GFP | RAGE | pEGFP-N1 | Xhol | Kpnl |
| RAGE-FLAG | FLAG | RAGE | p3xFLAG- | EcoRI | Kpnl |
| | | | CMV-14 | | |
| RAGE-RFP | RFP | RAGE | pRFP-N1 | EcoRI | Kpnl |

TABLE 2
Information of primers

| Primers | Sequences (5′ -> 3′) | SEQ ID NOS |
|---|---|---|
| P301L F | gat aat atc aaa cac gtc ctg gga ggc ggc ag | 3 |
| P301L R | ctg ceg cct ccc agg acg tgt ttg ata tta tc | 4 |
| G82S F | cgt gtc ctt ccc aac agc tec ctc ttc ctt cc | 5 |
| G82S R | gga agg aag agg gag ctg ttg gga agg aca cg | 6 |
| Cyto F | ggg gtc atc ttg tgg ggg gta cca gtc gac | 7 |
| Cyto R | gtc gac tgg tac ccc cca caa gat gac ccc | 8 |
| ΔV F | cgg aag gaa gag gga acc tac tac tgc ccc | 9 |
| ΔV R | ggg gca gta gta ggt tec ctc ttc ctt ccg | 10 |
| ΔC1 F | cag tgt gaa gag ccc ctt ccc agg aat ctg | 11 |
| ΔC1 R | cag att cct ggg aag ggg ctc ttc aca ctg | 12 |
| ΔC2 F | tat ctc agg gag gat ctg gat ggg ggc tgt | 13 |
| ΔC2 R | aca gcc ccc atc cag atc ctc cct gag ata | 14 |
| targeted | agt gtc ctc agg tcg ggt ga | 15 |
| allele F | | |
| targeted | cca tct aag tgc cag cta agg gtc | 16 |
| allele R | | |

### Example 11: Primary Cultures of Neurons, Microglia, and Astrocytes

The primary cortical and hippocampal neurons of the present invention were prepared from embryonic day 16.5. The neurons were plated on culture plates or microfluidic chamber devices coated with poly-L-lysine (Sigma-Aldrich) and maintained in Neurobasal medium (Gibco) containing 2% B-27 additive (Gibco), 100 U/ml penicillin-streptomycin, 10 μg/ml gentamicin and GlutaMAX additive (Gibco). Culture media were changed every 3 days and experiments were performed in vitro (DIV) on day 7. Primary microglia and astrocytes were prepared from the cortex of a postnatal day 1 pup, and maintained in DMEM medium containing 10% FBS, 100 U/ml penicillin-streptomycin, and 10 μg/ml gentamicin. Culture media were changed every 3 days and experiments were performed at DIV 14.

### Example 12: Cell Binding Assay and Calculation of Dissociation Constant (Kd)

The purified tau monomers of the present invention were biotinylated using the Ez-Link Sulfo-NHS-LC-Biotinylation kit (Thermo Scientific) and then fibrillated as described above to form oligomers and fibrils. SH-SY5Y cells were transfected with RAGE cDNA for 24 hours and incubated with biotin-tau protein for 2 hours. Primary cortical neurons from WT and Rage KO mice (DIV 7) were then incubated with various concentrations of biotin-tau oligomers for 24 hours to estimate the K_{d} value for tau binding to RAGE. Cells were washed with Tris-buffered saline (TBS) and fixed with 4% paraformaldehyde for 20 min. The cells were then blocked with 10% FBS and 0.1% Triton X-100 in TBS for 1 hour and incubated with streptavidin-alkaline phosphatase conjugate (1:2000, Roche) at 4° C. for 16 hours. After washing with TBS, bound biotin-tau was visualized for 10 min using the BCIP/NBP liquid substrate system (Sigma-Aldrich). Images were acquired using INCell Analyzer 2000, and cell-bound biotin signals were measured using Image J. K_{d} values estimated for RAGE-binding were obtained from nonlinear regression analysis of saturated binding using Prism (GraphPad Software).

### Example 13: In Vitro Co-Immunoprecipitation Assay

The HEK293T cell lysates in the present invention were prepared in lysis buffer (50 mM Tris-Cl pH 7.4, 150 mM NaCl, 1 mM EDTA, 1% Triton X-100) containing 1 mM PMSF (USB). After centrifugation at 13,000 rpm for 20 min at 4° C., the supernatant was diluted to the final concentration of 0.2% Triton X-100 using PBS, and incubated with 250 nM His-tagged tau protein overnight at 4° C. Samples were incubated with Ni-NTA agarose for full down assay or incubated with Sepharose 4 Fast Flow (GE Healthcare) along with anti-GFP antibodies overnight at 4° C. for 6 hr. After washing with PBS, samples were eluted in SDS-PAGE sample buffer containing 2-mercaptoethanol, and immunoblotting was performed after SDS-PAGE.

### Example 14: Tau Infection in Mouse Prefrontal Cortex

The present inventors anesthetized 3-month-old WT and Rage KO mice and injected 2.5 μl of PBS-soluble rTg4510 brain extract containing 1 g/ml human tau into the prefrontal cortices using a 30-gauge Hamilton microinjector (stereoscopic coordinates: anteroposterior (AP)=1.3 mm, mediolateral (ML)=1.5 mm, dorsoventral (DV)=−1.6 mm at the bregma). After 48 hours, the above mice were anesthetized and perfused with PBS containing 10 U/ml heparin and 4% paraformaldehyde. Brain sections (40 μm) were prepared from the prefrontal cortices and processed for immunohistochemistry. Images were obtained using a confocal laser scanning microscope LSM700, and human tau-positive cells were counted in the injection area.

### Example 15: Preparation of Recombinant Adenoviruses

The present inventors prepared recombinant adenoviruses and adeno-associated viruses (AAVs). The recombinant adenoviruses were prepared using methods known in the art (H. Park, et al., Hum. Mol. Genet. 21, 2725-2737, 2012). Specifically, GFP-tau was subcloned into pShuttle-CMV and transformed into BJ5183 cells using the pAdEasy-1 adenoviral backbone vector for homologous recombination. The recombinant AAV vectors were prepared by subcloning GFP-P301L tau into pJDK viral vector (courtesy of Dr. Hee-Ran Lee, Ulsan National University College of Medicine). Viruses were then produced using HEK293T cells and monitored by fluorescence microscopy. Cells were harvested and lysed by freeze-thawing, and viral particles were purified by CsCl gradient centrifugation or using the AAVpro purification kit (Takara Bio) according to the manufacturer's instructions. The concentration of infectious viral particles was estimated by infecting cells with a series of viral dilutions and counting GFP-positive cells using BD FACSCanto II (BD Biosciences).

### Example 16: Dot Blot Analysis

The primary hippocampal neurons (DIV 7) of the present invention were transduced with GFP-tau adenovirus (0.76×10⁸ TU/ml, MOI 10) for 24 hours or incubated with 500 nM tau oligomers for 24 hours. After 48 hours, cell lysates were prepared in 1% SDS lysis buffer and filtered through a 0.2 μm nitrocellulose membrane using a 96-well vacuum dot blot device (Bio-Rad). The membrane was stained with Ponceau S (USB) to indicate total protein loading, washed with TBS containing 0.05% Tween 20 (TBS-T), blocked with 5% nonfat milk in TBS-T for 1 hr, and analyzed by immunoblotting.

### Example 17: Tau Propagation Analysis

The present inventors performed tau propagation assays using a three-chamber microfluidic device. The microfluidic device was prepared by fabricating poly (dimethylsiloxane) (Sylgard 184, Dow Corning) in a master mold (courtesy of Dr. Nuri Jeon, Seoul National University), and bonded with poly-L-lysine-coated slides (J. W. Park, et al., Nat. Protoc. 1:2128-2136, 2006). Primary hippocampal neurons from WT and Rage KO mice were cultured in three chambers of the above apparatus: WT neurons in the first chamber, WT or Rage KO neurons in the second and third chambers, respectively. The WT neurons in the first chamber (DIV 7) were transduced with GFP-tau adenovirus (0.76×108 TU/ml, MOI 50) for 24 hours. The chambers were fluidically isolated from each other by setting a 50 μl volume difference to limit diffusion throughout the chamber. After 14 days, the neurons in the chambers were washed with PBS, fixed with 4% paraformaldehyde, and nuclei were visualized with a Hoechst 33342 (Sigma-Aldrich). Images were obtained using a fluorescence microscope (Olympus), and tau propagation through the chambers was compared by measuring intracellular GFP intensities in the chambers using Image J.

### Example 18: Purification and Stereotaxic Injection of Tau PHF in AD Brain (AD-Tau)

The AD-tau of the present invention was purified from human brain tissue from an AD patient (Harvard Brain Tissue Resource Center, McLean Hospital, Massachusetts) (J. L. Guo. et al., J. Exp. Med. 213:2635-2654, 2016). Two grams of the tissue was homogenized using a Dounce homogenizer in high salt buffer (10 mM Tris-HCl [pH 7.4], 0.8 M NaCl, 1 mM EDTA, 2 mM dithiothreitol [DTT], containing 0.1% sarcosyl and 10% sucrose, a protease inhibitor cocktail and a phosphatase inhibitor). The supernatant was filtered and additional sarcosyl was added to reach 1%, and it was then incubated for 1 hour at room temperature. The 1% sarcosyl-insoluble pellet containing pathological tau was collected after centrifugation at 4 ion 300,000×g for 1 hour and then resuspended in PBS by passing through a 27G needle. After resuspending the pellet and the resuspended pellet was sonicated (0.5 s pulses on/off) for 10 s (Branson Digital Sonifier, Danbury, CT) and further centrifuged at 100,000×g for 30 min at 4° C. further centrifuged to remove debris, and the supernatant containing concentrated AD PHF was used as AD-tau. Four-day-old C57BL/6 WT or RAGE KO mice were deeply anesthetized with a mixture of ketamine (100 mg/kg) and xylazine (10 mg/kg). PBS or AD-tau (4 μg/site) was injected unilaterally into the dorsal hippocampi and superior cingulate cortices using the following coordinates (stereotaxic coordinates: anteroposterior (AP)=−2.5 mm, mediolateral (ML)=+2.0 mm, dorsoventral (DV)=−2.4 mm and −1.4 mm from the bregma, respectively). After injection, the needle was retained for an additional 5 min to ensure complete absorption of the solution and the mice were monitored. For immunohistochemical analysis 6 months after injection, the mice were perfused with PBS and 4% PFA, and the brains were excised, fixed in 4% PFA overnight, and transferred to 30% sucrose solution for cryoprotection.

### Example 19: In Vivo Tau Propagation Using an AAV System

The present inventors anesthetized 3-month-old WT and Rage KO mice and injected 5 μl GFP-P301L tau AAV (6.5×10¹⁰ ifu/ml) into the left hippocampi using a 30-gauge Hamilton microinjector (stereoscopic coordinates: anteroposterior (AP)=−2.1 mm, mediolateral (ML)=1.8 mm, and dorsoventral (DV)=−2.0 mm in the bregma). After 20 weeks, the mice were analyzed with the Y-maze, novel recognition, and passive avoidance tests. The mice were then anesthetized and perfused with PBS containing 10 U/ml heparin and 4% paraformaldehyde, and the brain sections (40 μm) were prepared from the hippocampi and processed for immunohistochemistry. Images were obtained using a confocal laser scanning microscope LSM700, and tau propagation was assessed by measuring the signal intensities of GFP and human oligomeric tau in the hippocampi.

### Example 20: Behavioral Test

Two-month-old non-transgenic or tTA-negative mice and their littermates with rTg4510 used in the present invention were intraperitoneally injected with vehicle (5% DMSO) or RAGE antagonist (1 mg/kg/day) daily for 2.5 months. The mice were then analyzed by the Y-maze, novel object recognition and passive avoidance tests. In the Y-maze test, the mice were placed at the end of one arm of a Y-shaped maze (32.5 cm long×15 cm high) and allowed to move freely for 7 min. Entry into the arm was counted when the entire body, including the tail, was positioned in the arm. The percentage of spontaneous changes was estimated as the ratio of the number of changes to the total number of items. For the novel object recognition test, the mice were placed in a chamber (30 cm long×30 cm wide×25 cm high) and allowed to move freely for 7 min at 24 hour intervals. Prior to the test period, the mice were acclimatized to an empty chamber for 2 days. During the 3-day experimental period, two objects were placed in the chamber, one of which was replaced daily (novel object) and the other was left unchanged (familiar object). Object exploration was defined as a mouse sniffing the object or touching the object with its nose. The object discrimination index was estimated as the ratio of novel object exploration time to total object exploration time. For the passive avoidance test, a device with a light and dark compartment (20×20×20 cm each) separated by a sliding door was used for testing. The mice were placed in the closed light compartment and allowed to move freely for one minute before the door was opened. For conditioning, the latency of the mice to enter the dark compartment was measured, and an electric shock (0.25 mA, 2 s) was delivered by a floor grating after the door was closed. For testing, the mice were placed in a light compartment that was closed for 24 hours after conditioning and allowed to move freely for 1 minute before the door was opened, and latency to enter the dark compartment was measured with a 5-minute cutoff.

### Example 21: Immunoblotting Assay

For the extraction of nuclei, the present inventors resuspended cell pellets in hypotonic buffer (20 mM Tris-Cl pH 7.4, 10 mM NaCl, 3 mM MgCl₂, 0.5% NP-40) containing 1 mM PMSF. The supernatant was then centrifuged at 3,000 rpm for 10 min at 4° C. and the supernatant was separated into a cytoplasmic fraction. A nucleus fraction was prepared by sonication of the pellets in a cell extraction buffer (10 mM Tris-Cl pH 7.4, 100 mM NaCl, 1% Triton X-100, 0.1% SDS, 0.5% sodium deoxycholate, 10% glycerol, 1 mM EDTA) containing 1 mM PMSF. After centrifugation at 13,000 rpm for 20 min at 4° C., the supernatant was separated as a nucleus fraction. The cell lysate was prepared by sonication in a lysis buffer (50 mM Tris-Cl pH 8.0, 150 mM NaCl, 1% Triton X-100, 0.1% SDS, 0.5% sodium deoxycholate, and 1 mM EDTA) containing 1 mM PMSF. After centrifugation at 13,000 rpm for 20 min at 4° C., the supernatant was separated by SDS-PAGE and transferred to a PVDF membrane (ATTO Corporation). The blot was then blocked with 5% BSA in TBS-T for 1 hour and incubated with primary antibodies in TBS-T overnight at 4° C. After washing with TBS-T, the blot was incubated with peroxidase-conjugated secondary antibody (Jackson ImmunoResearch Laboratories: 1:40,000) for 1.5 hours and visualized using an ECL detection system. Information about the antibodies used in the present invention is summarized in Table 3 below.

TABLE 3
Information of used antibodies

| Antibodies, | | Catalog | | |
|---|---|---|---|---|
| Clones | Source | Number | Host | Apply |
| β-actin, C4 | Santa Cruz | sc-47778 | Mouse | 1:3000 (IB) |
| FLAG, M2 | Sigma-Aldrich | F1804 | Mouse | 1:5000 (IB) |
| GFAP | Millipore | MAB5628 | Mouse | 1:500 (ICC) |
| GFP, B-2 | Santa Cruz | sc-9996 | Mouse | 1:3000 (IB) |
| | | | | 1:500 (IP) |
| His-tag | Cell Signaling | 2365 | Bunny | 1:1000 (IB) |
| lba1 | Wako | 019-19741 | Bunny | 1:500 (ICC) |
| Lamin A/C, | Santa Cruz | sc-376248 | Mouse | 1:3000 (IB) |
| E-1 | | | | |
| MAP2 | Cell Signaling | 4542 | Bunny | 1:500 (ICC, |
| | | | | IHC) |
| MAP2B, 18 | BD | 610460 | Mouse | 1:500 (ICC) |
| | Biosciences | | | |
| NFκB p65 (C-20) | Santa Cruz | sc-372-G | Goat | 1:3000 (IB) |
| RAGE | Invitrogen | PA5-78736 | Bunny | 1:3000 (IB) |
| RAGE(N-16) | Santa Cruz | sc-8230 | Goat | 1:500 (IHC) |
| Tau, TG5 | Dr. Peter | | Mouse | 1:1000 (IB) |
| | Davies | | | |
| Human tau, HT7 | Invitrogen | MN1000 | Mouse | 1:500 (ICC, |
| | | | | IHC) |
| | | | | 1:10000 (IB) |
| Oligomeric tau | Merk | ABN454 | Bunny | 1:5000 (IB) |
| (T22) | | | | |
| Phospho-Tau | Invitrogen | MN1020 | Mouse | 1:500 (IHC) |
| (Ser202, Thr205), | | | | |
| AT8 | | | | |

### Example 22: Immunocytochemistry and Immunohistochemistry

For immunocytochemistry, the present inventors washed cells with PBS and fixed with 4% paraformaldehyde for 20 min, and after washing with PBS, the cells were blocked with 3% BSA in PBS for 1 hour and incubated overnight at 4° C. with primary antibodies in PBS containing 1% BSA. The cells were then incubated with Alexa Flour 488 or 594 secondary antibodies (Jackson ImmunoResearch Laboratories; 1:500) for 1.5 hours, and the nuclei were visualized with Hoechst 33342. Coverslips were placed on slides with mounting medium (Sigma-Aldrich). For immunohistochemistry, brain sections (40 μm) were prepared, washed in PBS and blocked with 10% FBS in PBS containing 1% Triton X-100 for 1 hour. Then, they were incubated with primary antibodies in PBS containing 5% FBS and 0.1% Triton X-100 overnight at 4° C. After washing, the Claims were incubated with Alexa Flour 405, 488, or 594 secondary antibodies (Jackson ImmunoResearch Laboratories; 1:500) for 1.5 hours, and the nuclei were visualized with a Hoechst 33342. Claims were placed on slides with mounting medium.

### Experimental Example 1: Cell-Based Tau Infection Assay

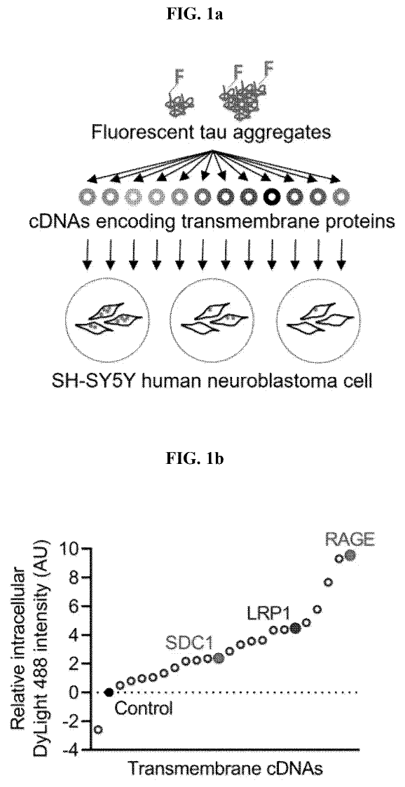

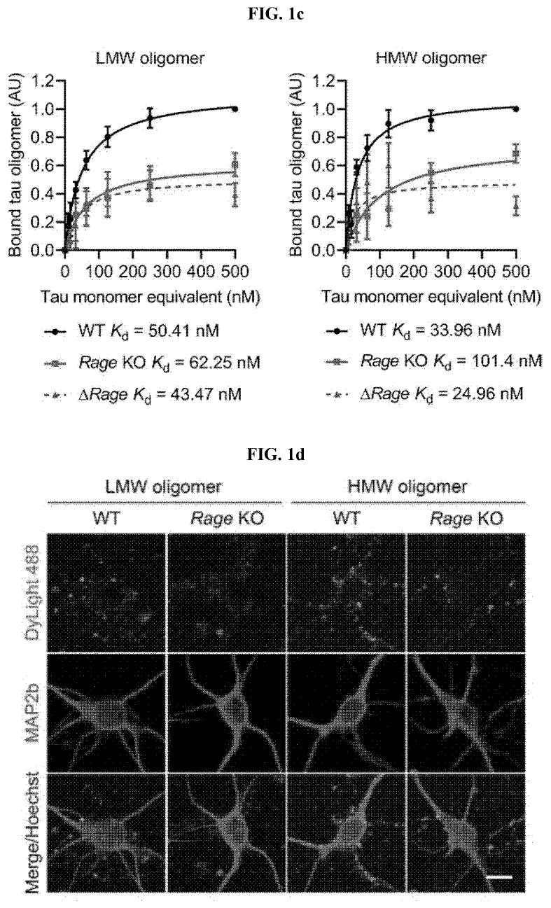

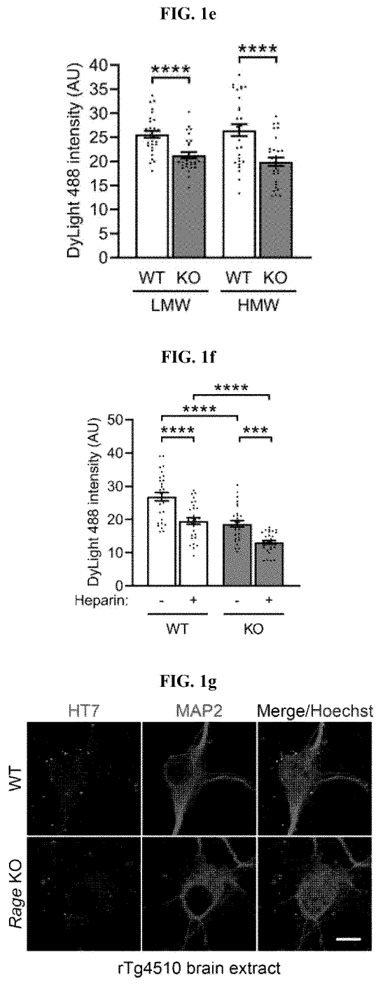

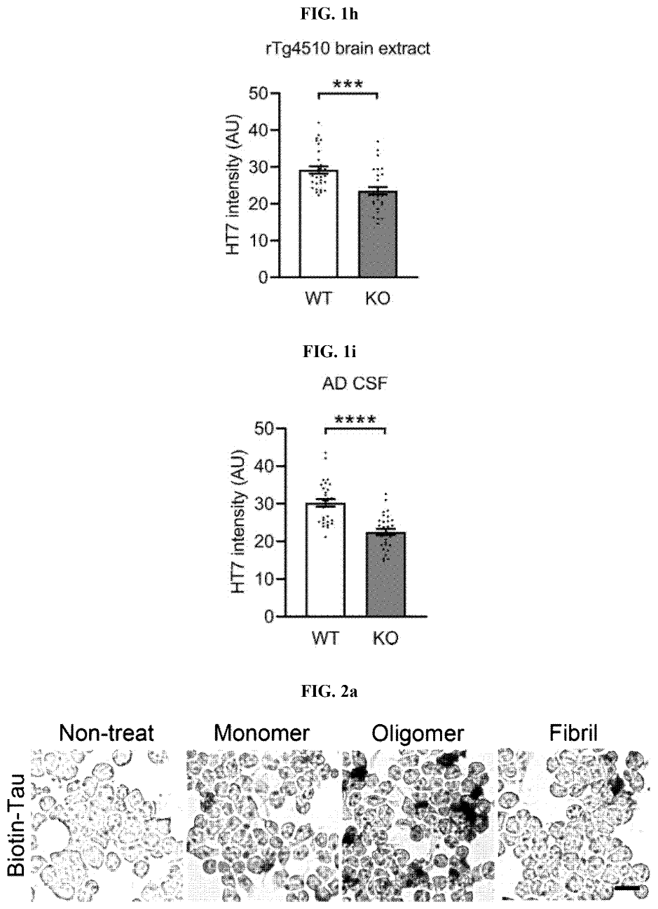

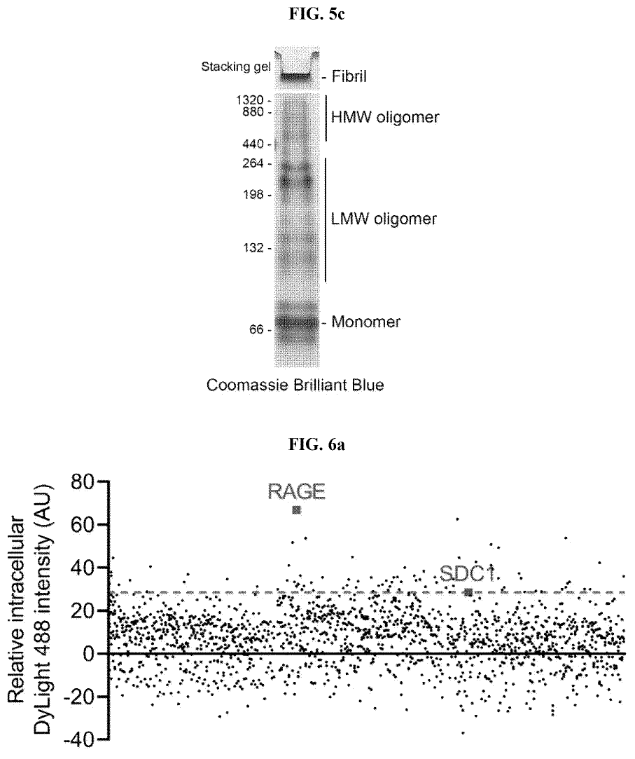

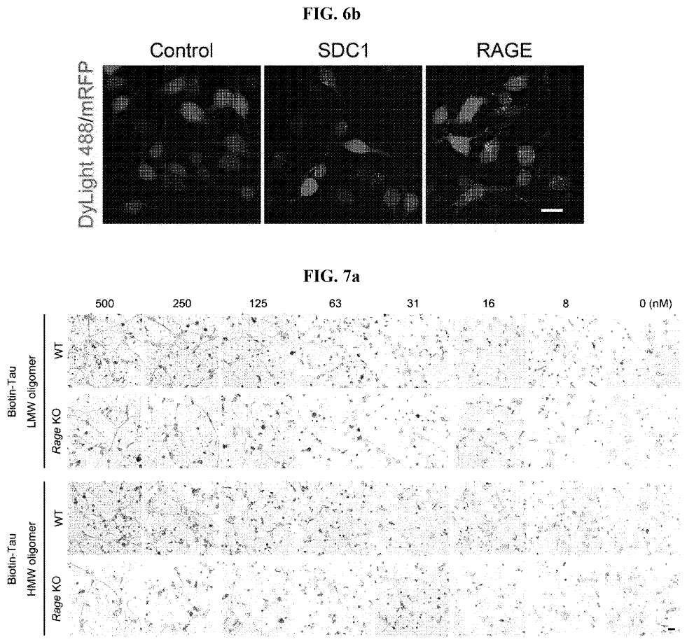

The present inventors performed a cell-based tau transfection assay to identify the membrane receptor responsible for neuronal propagation of tau oligomers. For this purpose, purified His-tagged human tau protein was labeled with DyLight 488 (DyLight 488-tau), and formed aggregates after incubation with heparin. Using high-speed protein liquid chromatography (FPLC), it was found that the tau aggregates were generated mainly in oligomeric (10-20 units) and fibrillary forms (FIGS. 5a and 5c). Mammalian expression vectors comprising cDNA encoding each full-length human and mouse transmembrane protein (1,523 in total) were obtained, and for analysis, SH-SY5Y human neuroblastoma cells were transfected with each cDNA and incubated with DyLight 488-tau aggregates (FIG. 1a). Using the above approach, the present inventors isolated a list of positive clones that presumably enhance the intracellular infection of tau aggregates (FIGS. 6a and Table 4). Among them, RAGE most efficiently mediated the internalization of tau protein by the transfected cells (FIGS. 1b and 6b). The present inventors further classified the tau oligomeric species into low molecular weight (LMW, 2-4 units) and high molecular weight (HMW, 10-20 units) using FPLC (FIGS. 5b and 5c). Incubation of primary cultured wild-type (WT) and Rage knockout (KO) neurons with tau oligomers showed that cell binding and uptake of LMW and HMW tau oligomers by Rage KO neurons was significantly reduced compared to that by WT neurons (FIGS. 1c to e and 7a). HSPG-mediated macrocytosis has recently been highlighted as a mechanism for cellular tau infection (J. N. Rauch, et al., Nature. 580:381-385, 2020). Indeed, antagonizing HSPG using heparin reduced neuronal uptake of tau oligomers into WT neurons (FIGS. 1f and 7b). Furthermore, tau oligomer transfection into Rage KO neurons was efficiently blocked by heparin (FIGS. 1f and 7b), indicating that the RAGE-mediated neuronal uptake of tau protein is independent of HSPG. Genes identified as the regulators of the intracellular transfection of tau aggregates are summarized in Table 4 below.

TABLE 4
Information of genes

| Intracellular DyLight 488 | | |
|---|---|---|
| Intensities (AU) | Genes | Access Number |
| 9.55 | RAGE | NM_001136 |
| 9.30 | PEX11A | NM_003847 |
| 7.66 | GHITM | NM_014394 |
| 5.78 | MFN1 | NM_033540 |
| 4.86 | GJB3 | NM_024009 |
| 4.47 | LRP1 | NM_008512 |
| 4.36 | SACM1L | NM_001319071 |
| 4.32 | SLC25A37 | XM_011544554 |
| 3.62 | CD47 | NM_001777 |
| 3.56 | SLC25A5 | NM_001152 |
| 3.33 | LMBRD1 | NM_018368 |
| 2.86 | F2RL1 | NM_005242 |
| 2.38 | SDC1 | NM_002997 |
| 2.35 | LAPTM4A | NM_014713 |
| 2.23 | SLC25A32 | NM_030780 |
| 2.16 | ADGRA3 | NM_145290 |
| 1.72 | MGST3 | NM_004528 |
| 1.33 | SLC2A12 | NM_145176 |
| 1.04 | CD63 | NM_001780 |
| 0.97 | CMTM3 | NM_144601 |
| 0.79 | SEC63 | NM_007214 |
| 0.49 | SLC23A2 | NM_005116 |

### Experimental Example 2: Neuronal Uptake of Tau Protein

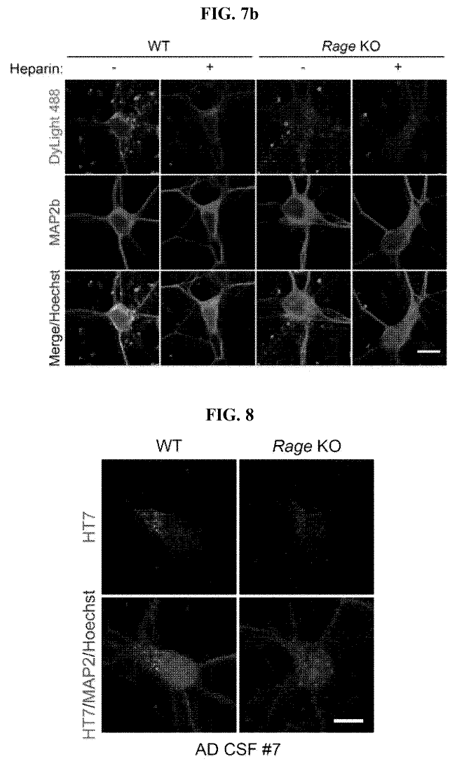

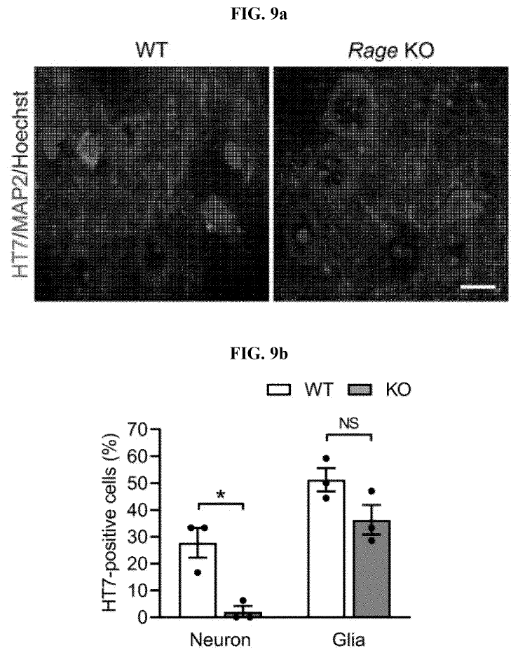

Given that many different tau species could be the subject of neuronal propagation (F. Clavaguera, et al, Proc. Natl. Acad. Sci. 110:9535-9540. 2013), the present inventors evaluated neuronal uptake of tau protein prepared from rTg4510 mice expressing human P301L mutant tau under the control of the Ca²⁺-calmodulin kinase 301CaMKII) promoter (K. SantaCruz, et al, Science. 309:476-481, 2005). When treated with PBS-soluble brain extracts from an rTg4510 mice (S. Takeda, et al. Nat. Commun. 6:8490, 2015), human P301L tau proteins were observed inside WT neurons but were significantly reduced in Rage KO neurons (FIGS. 1g and 1h). The present inventors also tested the uptake of tau species containing phospho-tau181 purified from cerebrospinal fluid (CSF) of AD patients (N. S. M. Schoonenboom, et al. Neurology. 78:47-54, 2012) (Table 5), and similarly, the neuronal infection of tau species in the AD CSF was activated in WT neurons but significantly impaired in Rage KO neurons (FIG. 1i and FIG. 8). Furthermore, the Rage deficiency blocked neuronal tau infection in vivo when the rTg4510 brain extracts were injected into the prefrontal cortices of WT and Rage KO mice (FIGS. 9a and 9b). Clinical information about the AD CSF samples used in the present invention is summarized in Table 5 below.

TABLE 5
Toxic genes

| AD CSF | | Aβ42 | Total Tau | P-tau181 | Amyloid |
|---|---|---|---|---|---|
| number | Analytics | (pg/ml) | (pg/ml) | (pg/ml) | PET Reading |
| 7 | LOAD | 356.4 | 1,265.7 | 128.4 | Positive |
| 8 | LOAD | 222.3 | 516.6 | 66.8 | Positive |
| 9 | EOAD | 467 | 567.2 | 85.9 | Positive |

### Experimental Example 3: Tau Infection into Cells

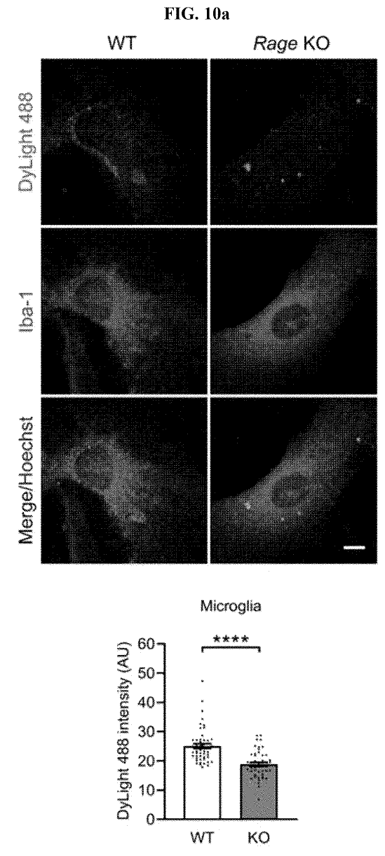

Because RAGE is also expressed in microglia and astrocytes (L.-F. Lue, et al., Exp. Neurol. 171:29-45, 2001), the present inventors tested whether RAGE mediates tau infection into these cells. As a result, the present inventors found that both primary cultured WT microglia and astrocytes readily internalize extracellular tau oligomers (FIGS. 10a and 10b). In microglia, Rage KO reduced tau oligomer uptake as seen in neurons (FIG. 10a). However, the transfection of tau oligomers into astrocytes was not affected by Rage deficiency (FIG. 10b). Thus, human P301L tau transfection into glial cells in the frontal cortices of Rage KO mice was reduced, but significant levels remained (FIGS. 9a and 9b). These results suggest that RAGE promotes the intracellular infection of pathologic tau species into neurons and microglia, but not astrocytes.

### Experimental Example 4: Interaction Between RAGE and Tau

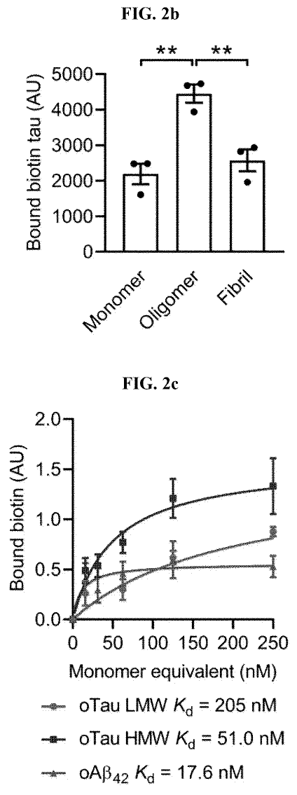

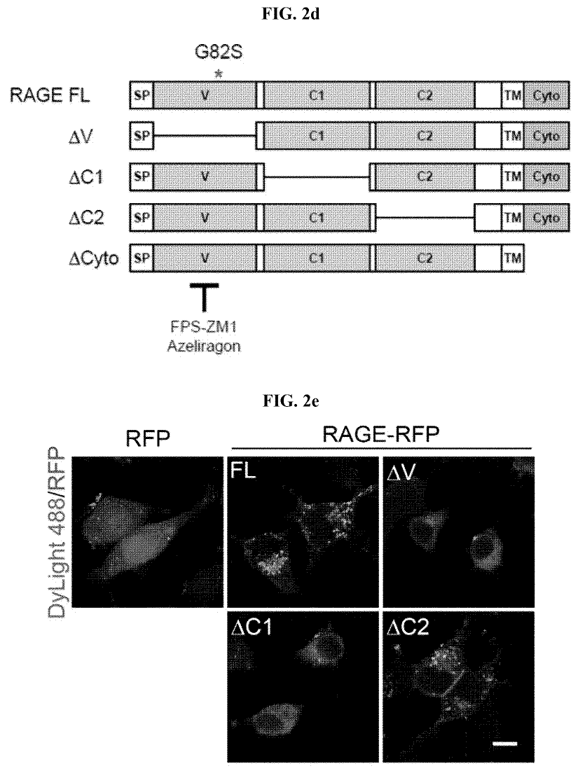

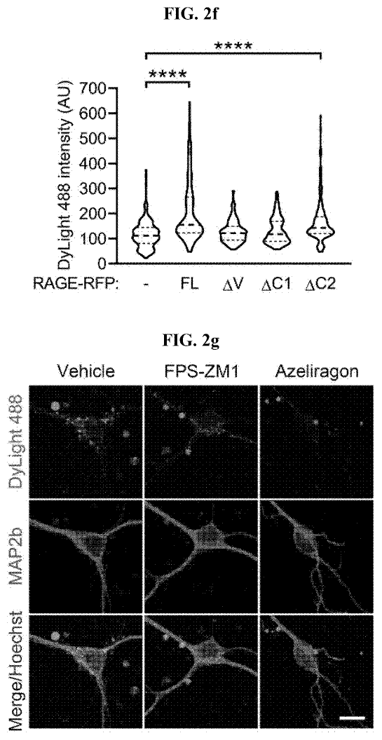

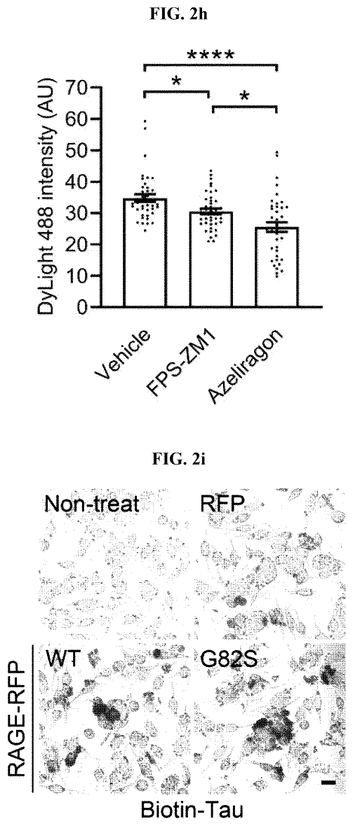

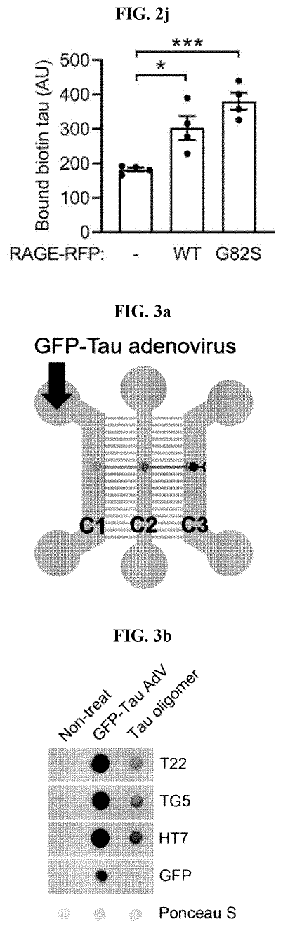

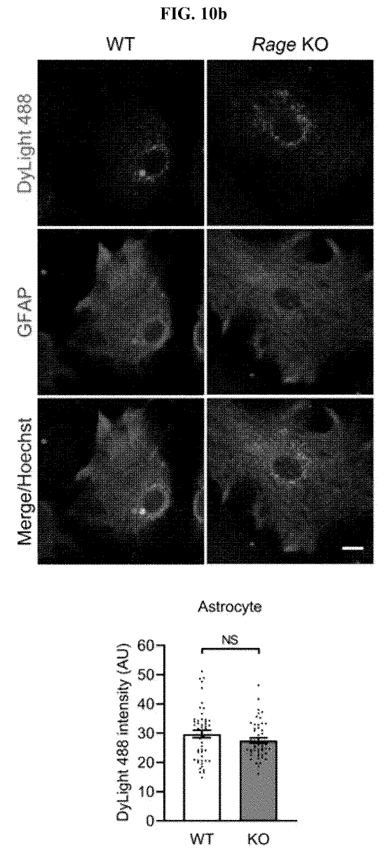

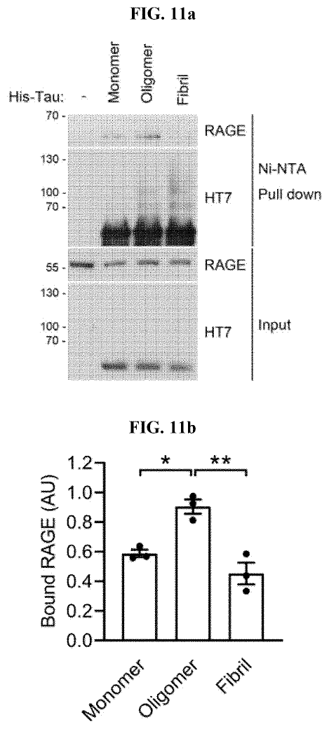

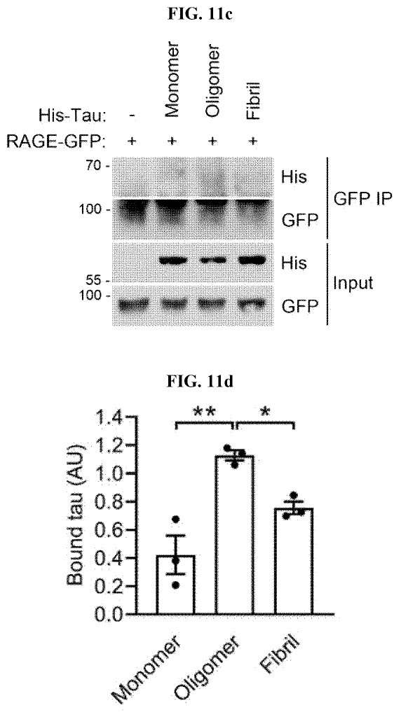

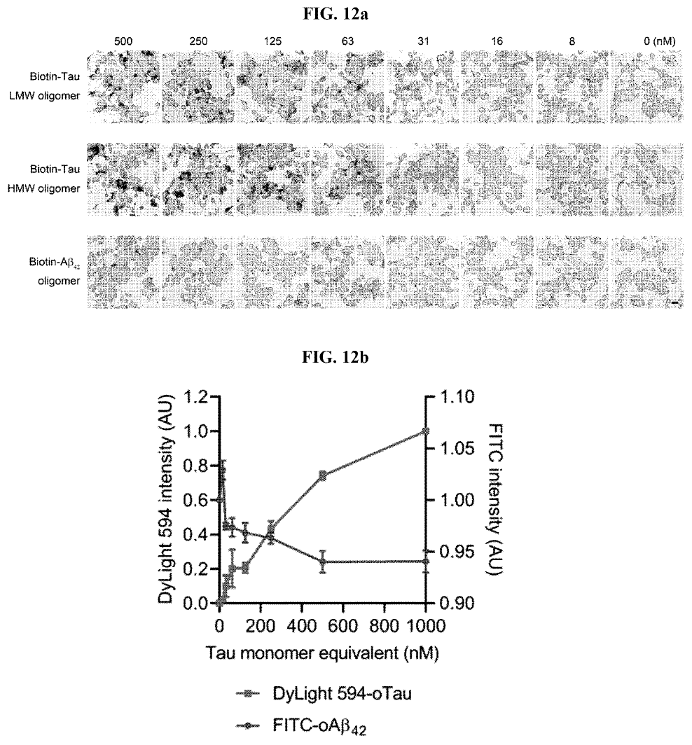

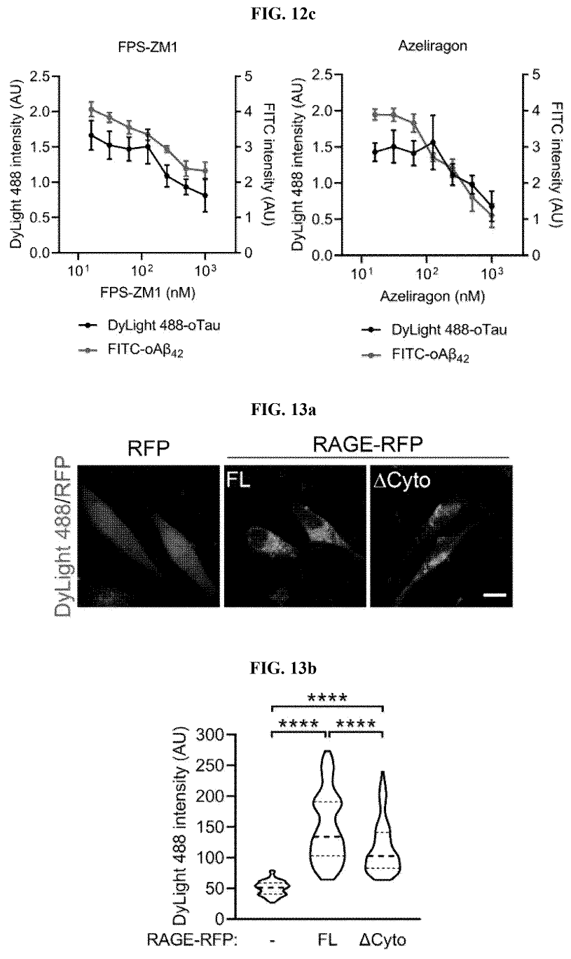

The present inventors prepared tau proteins in monomeric, oligomeric, and fibrillary forms and analyzed them for their interactions with RAGE. The results show that RAGE is bound to a variety of tau species but has a significant preference for oligomers (FIGS. 2a, 2b and 11). Because RAGE is also known to interact with amyloid beta (Aβ) (S. D. Yan, et al., Nature. 382:685-691, 1996), the present inventors compared RAGE interaction with tau oligomers to that with Aβ₄₂ oligomers (FIGS. 2c and 12a). The dissociation constant (K_{d}) for RAGE which binds to Aβ₄₂ oligomers was 17 nM monomer equivalents of total Aβ₄₂, while the dissociation constants (K_{d}) for LMW and HMW tau oligomers were 205 nM and 51 nM monomer equivalents of total tau, respectively (FIGS. 2c and 12a). Notably, when tau oligomers and Aβ₄₂ oligomers were treated simultaneously, an increase in tau oligomers efficiently reduced the intracellular transfection of Aβ₄₂ oligomers (FIG. 12b), indicating that their binding to RAGE is competitive.

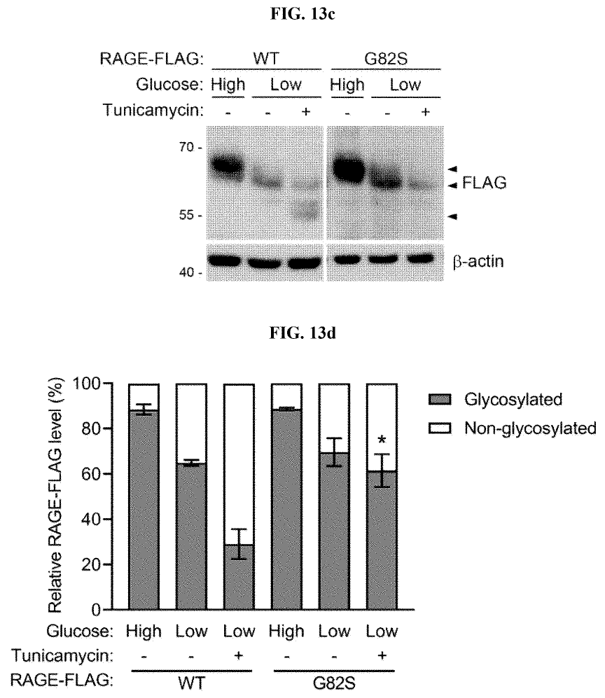

To further characterize the interaction between RAGE and tau oligomers, the present inventors also generated several RAGE mutants lacking extracellular domains (FIG. 2d). Overexpression of RAGE mutants in SH-SY5Y cells showed that deletion of the V or C1 domains reduced the intracellular infection of tau oligomers (FIGS. 2e and 2f). Thus, the RAGE V and C1 domains are required for the binding and internalization of tau oligomers. The RAGE mutant lacking the cytoplasmic domain (FIG. 2d) internalized tau oligomers, albeit with lower efficiency compared to the full-length form (FIGS. 13a and 13b). The present inventors then evaluated the effect on tau infection of FPS-ZM1 and Azeliragon, two RAGE antagonists that bind to the V domain and competitively inhibit the binding of RAGE ligands including Aβ₄₂ oligomers (R. Deane, et al., Clin. Invest. 122:1377-1392, 2012) (FIGS. 2d and 12c). The results showed that the FPS-ZM1 or Azeliragon treatment inhibited tau infection into RAGE-expressing SH-SY5Y cells (FIG. 12c) and neurons (FIGS. 2g and 2h). Importantly, the G82S polymorphism of RAGE in the V domain is associated with increased AD susceptibility (K. Li, et al., J. Neural Transm. 117:97-104, 2009) and promotes glycosylation of RAGE (FIG. 2d), resulting in structural changes and enhanced ligand binding (FIGS. 13c and 13d) (M. A. Hofmann, et al., Genes Immun. 3:123-135, 2002). Notably, the G82S RAGE exhibited increased tau binding compared to the WT RAGE (FIGS. 2i and 2j), suggesting an effect of RAGE glycosylation on tau binding and infection.

### Experimental Example 5: Evaluation of the Role of RAGE

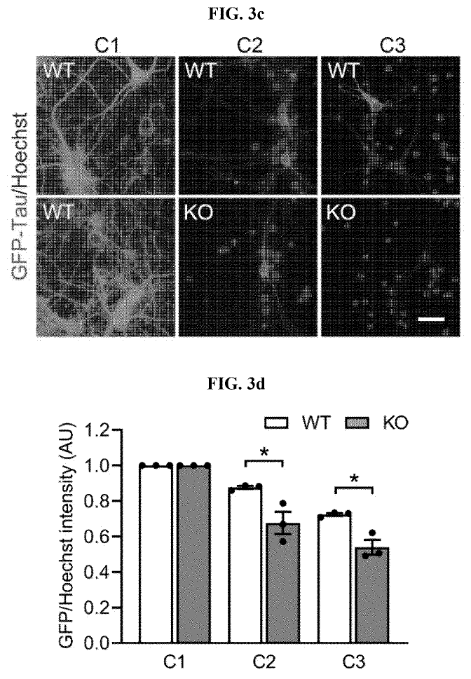

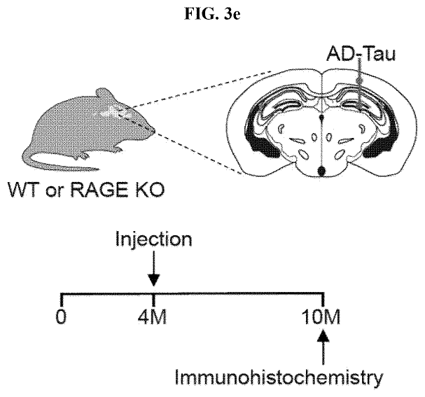

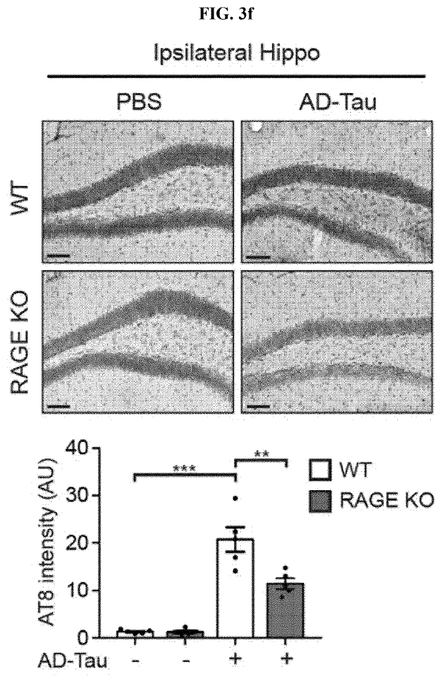

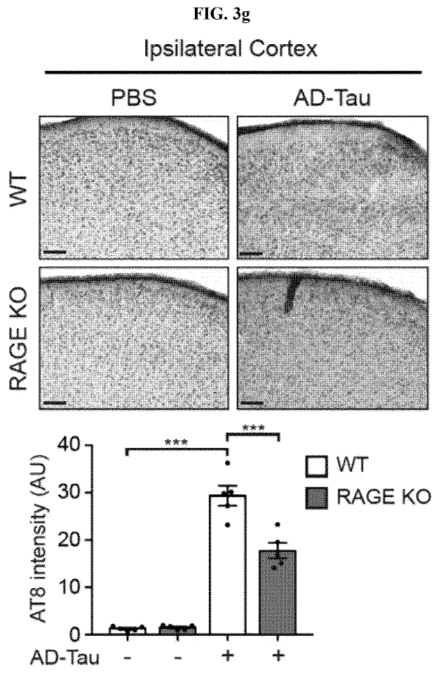

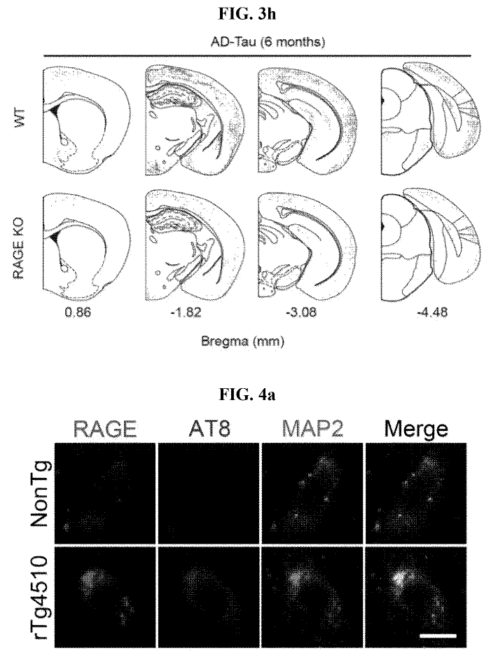

To assess the role of RAGE in transsynaptic tau propagation, the present inventors used a three-chamber microfluidic device that allows the observation of neuron-to-neuron transmission via axonal extension in a chamber (J. W. Park, et al., Nat. Protoc. 1:2128-2136, 2006). For the above analysis, primary hippocampal neurons were cultured in three chambers: WT neurons in the first chamber (C1), WT or Rage KO neurons in the second and third chambers (C2 and C3) (FIG. 3a). The present inventors then used adenovirus to overexpress GFP-tau in C1 neurons for seed aggregation and assessed GFP-tau propagation from C1 to C2 and C3. Using dot blot analysis, the present inventors confirmed robust formation of detergent-insoluble tau aggregates within the neurons transduced with GFP-tau adenovirus compared to the neurons treated with tau oligomers (FIG. 3b). Fourteen days after adenoviral transduction, the GFP-tau aggregates exhibited trans-synaptic propagation spread from C1 to C3 in WT neurons (FIG. 3c). In contrast, Rage deficiency reduced GFP-tau propagation in neurons by ˜20% (FIGS. 3c and 3d). The present inventors also tested the role of RAGE in tau propagation in vivo using tau fibrils prepared from the brains of AD patients (AD-tau), which contain minimal Aβ and drive the propagation of pathologic tau in non-transgenic (NonTg) mice (J. W. Park, et al., Nat. Protoc. 1:2128-2136, 2006) (FIG. 3e). After unilateral injection of AD-tau into the dorsal hippocampi and overlying cortices for 6 months, the present inventors detected AT8-positive pathologic tau aggregates in the hippocampi and cortices of WT mice (FIGS. 3f and 3g). On the other hand, the detection of AD-tau-seeded tau aggregates was significantly lower in the brains of Rage KO mice than in WT mice (FIGS. 3f and 3g). Furthermore, tau propagation to other brain regions, such as the entorhinal cortex, corpus callosum, and mammillary region, was active in WT mice but was significantly reduced by Rage deficiency (FIG. 3h). These results suggest that RAGE is an important mediator of neurotransmission of pathogenic tau forms.

### Experimental Example 6: Analysis of RAGE Expression Pattern

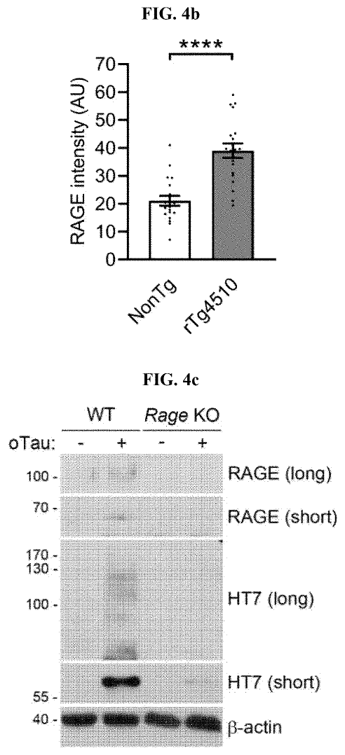

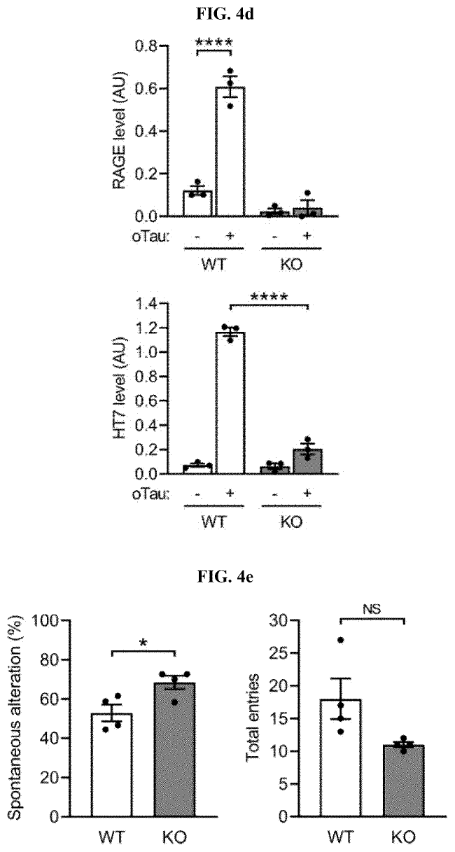

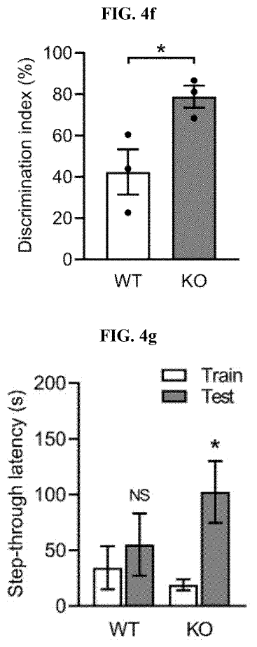

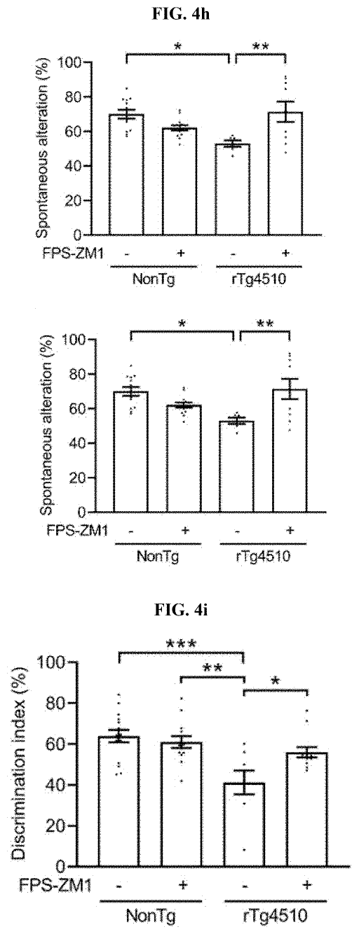

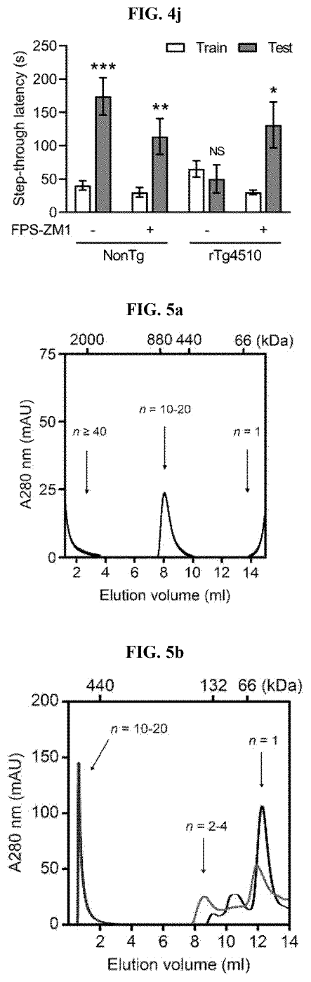

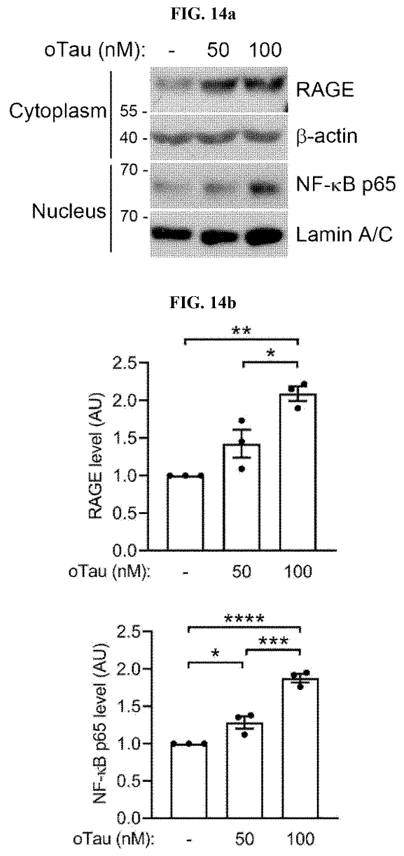

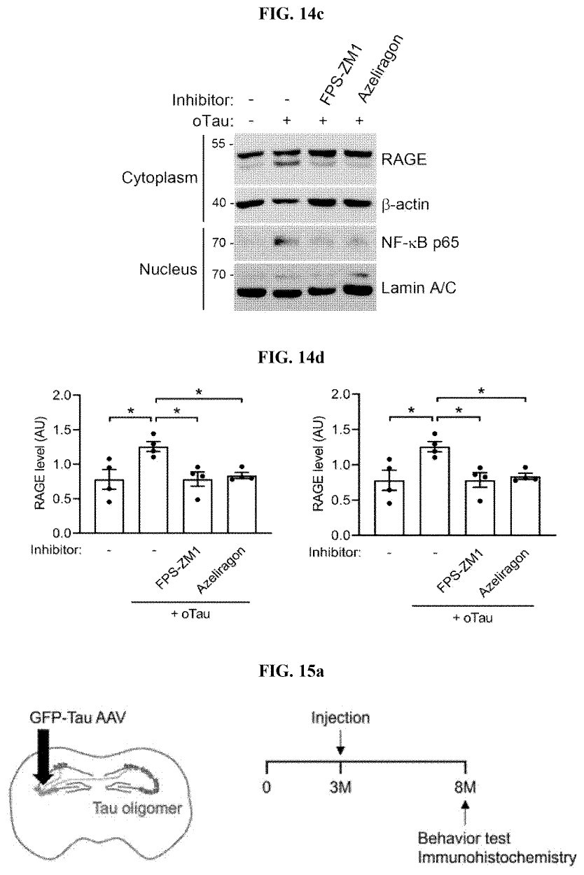

To further investigate the role of RAGE in tau pathogenesis, the present inventors analyzed RAGE expression patterns in the brains of rTg4510 mice expressing human P301L tau, which is largely restricted to forebrain structures (K. SantaCruz, et al., Science. 309:476-481, 2005). The results showed that RAGE expression in hippocampal neurons was increased in rTg4510 mice than in age-matched NonTg mice (FIGS. 4a and 4b). Furthermore, RAGE expression was increased up to 5-fold in primary cultured neurons upon tau oligomer treatment (FIGS. 4c and 4d). As RAGE-ligand binding is known to activate the MAPK/NF-κ pathway and induce the expression of RAGE itself (K. Kierdorf, et al. J. Leukoc. Biol. 94:55-68, 2013), tau oligomers also induced nuclear translocation of NF-κ5 p65 (FIGS. 14a and 14b), which was blocked by disrupting RAGE antagonists and RAGE-tau bindings (FIGS. 14c and 14d). Thus, RAGE levels in neurons are upregulated by tau oligomers, which likely enhances tau propagation in a vicious cycle.

### Experimental Example 7: Behavioral Test

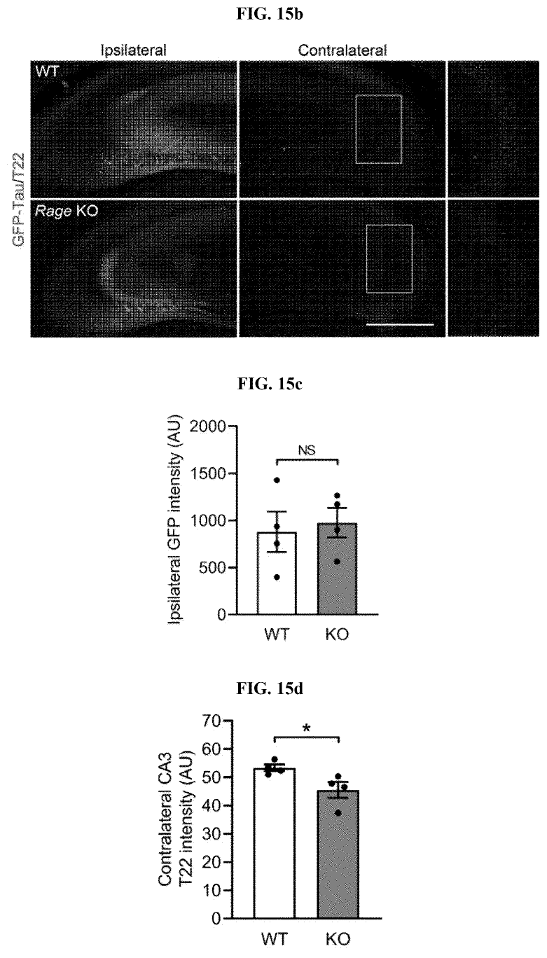

The present inventors constructed adeno-associated viruses for expression of GFP-tau with the P301L mutation (GFP-tau AAV) in the hippocampus and investigated whether RAGE participates in tau-induced behavioral deficits. To this end, behavioral tests were analyzed 5 months after unilateral GFP-tau AAV injection into the hippocampi of WT and Rage KO mice (FIG. 15a). The results showed that WT mouse exhibited a significant reduction in spatial memory in the Y-maze test (FIG. 4e), and learning and memory declines in the novel object recognition test (FIG. 4f) and passive avoidance test (FIG. 4g); however, Rage KO mouse did not exhibit the above cognitive impairments after GFP-tau expression (FIGS. 4e to 4g). When examining tau propagation, T22-positive tau immunoreactivity was found in the ipsilateral hippocampi adjacent to the injection sites and also in the contralateral hippocampi of the WT mice: however, Rage deficiency reduced T22 immunoreactivity by ˜20% in the contralateral CA3 regions (FIGS. 15b to 15d).

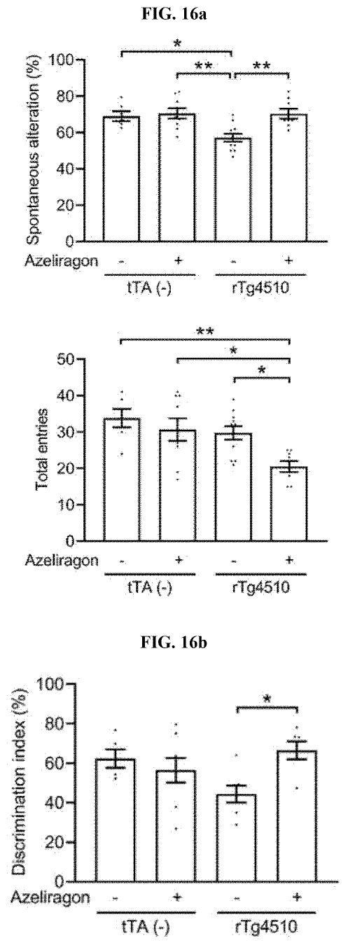

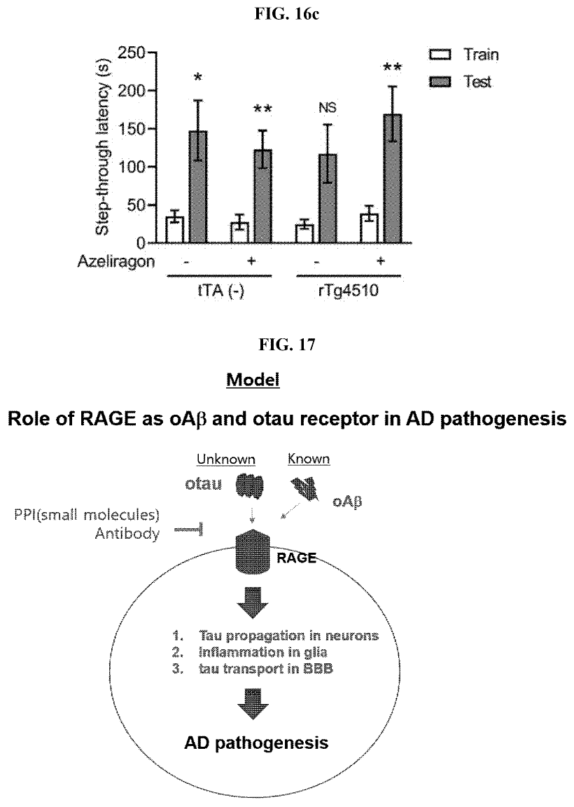

In addition, to assess cognitive function in rTg4510 mice, the present inventors tested whether blocking RAGE-tau interactions using RAGE antagonists could delay tau pathogenesis. rTg4510 mice exhibit tau pretangles within the cortex from 2.5 months of age, followed by progressive formation of tau inclusions within the hippocampus, resulting in memory decline as they age from 2.5 to 4.5 months of age (M. Ramsden, et al. J. Neurosci. 25:10637-10647, 2005). Therefore, the present inventors initiated administration of RAGE antagonists at 2 months of age and performed behavioral tests at 4.5 months of age. As a result, 4.5-month-old vehicle-treated rTg4510 mice exhibited cognitive deficits in the Y-maze test (FIG. 4h), novel object recognition test (FIG. 4i), and passive avoidance test (FIG. 4j). Notably, FPS-ZM1 or Azeliragon-injected rTg4510 mice exhibited significant retention of spatial and working memory (FIGS. 4h to 4j and FIG. 16). The above results suggest that RAGE is important in the progression of tau pathology and that blocking its function may delay memory impairment.

### Experimental Example 8: Inhibitory Effect of Antibodies

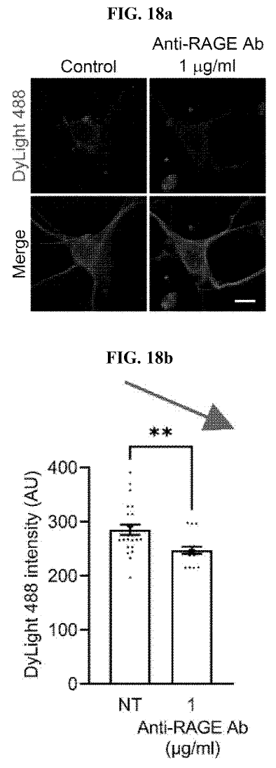

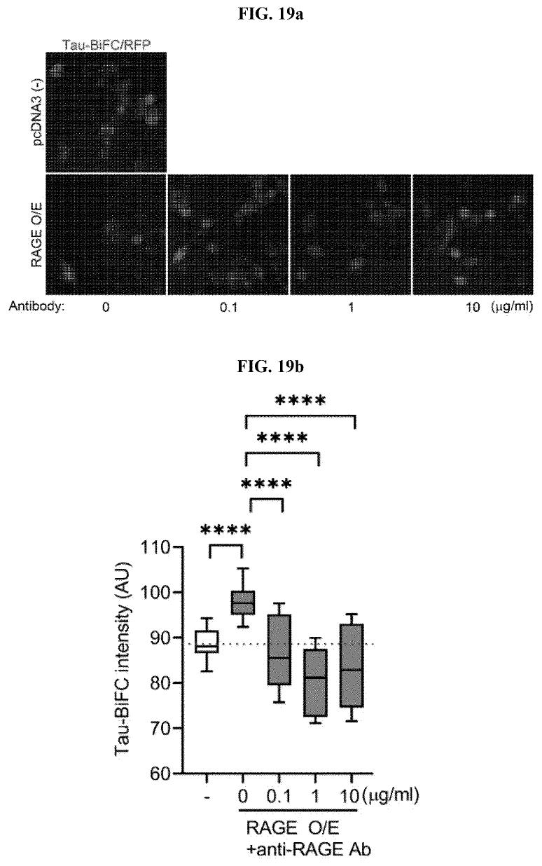

Furthermore, for the purpose of inhibiting RAGE progression by targeting the RAGE addressed in the present invention, antagonistic small molecule drugs targeting the V-C1 domain of RAGE, such as FPS-ZM1 or Azeliragon, or anti-RAGE antibodies targeting the V-C1 domain of RAGE can be utilized to block the protein-protein interaction between the tau oligomers and RAGE (FIG. 17). The present inventors treated WT hippocampal neurons with 500 nM DyLight 488-tau oligomers in the presence of 1 μi/ml anti-RAGE antibodies for 24 hours. After washing, the cells were immunostained with anti-MAP2 antibodies, and tau infection of the neurons was visualized. To evaluate the effect of anti-RAGE antibodies on the tau infection, primary cultured WT hippocampal neurons were incubated with tau oligomers, and the results showed that the uptake of tau oligomers by neurons was significantly reduced upon anti-RAGE antibody treatment (FIGS. 18a and 18b). Furthermore, to evaluate the effect of anti-RAGE antibodies on tau propagation between cells, SH-SY5Y cells expressing VN-tau and SH-SY5Y cells expressing tau-VC were co-cultured for 48 hours and tau propagation was confirmed by fluorescence arising from the binding of VN/VC. The results showed that the treatment with anti-RAGE antibodies in the tau-BiFC system inhibited the increased tau propagation according to RAGE overexpression in a dose-dependent manner (FIGS. 19a and 19b).

In conclusion, the present inventors investigated the pathogenesis of tau by focusing on the membrane receptors through which pathologic tau protein is transmitted between neurons. Because different tau strains are subjects for protein transmission, the present inventors reasoned that evaluating uptake of tau strains from different tauopathies would reveal different strain-specific receptors. The present inventors observed that RAGE binds to tau oligomers as efficiently as to Aβ oligomers, indicating that different protein aggregates may coincide at the central receptor. Thus, identifying mechanisms that are shared and distinct in different proteinopathies may contribute to a better understanding of these complex pathologies. The above results demonstrate that blocking RAGE function ameliorates tau-induced cognitive impairments. Therefore, the present invention can be utilized to develop therapeutics that target the neuronal tau propagation process in the early stages of tauopathies.

The present invention has been described with reference to the embodiments described above, but these are exemplary only, and one of ordinary skill in the art will understand that various modifications and other equally valid embodiments are possible from them. The true scope of technical protection of the invention should therefore be determined by the technical ideas of the appended claims of the patent.

### INDUSTRIAL AVAILABILITY

The pharmaceutical composition and drug screening methods according to one embodiment of the present invention can be very useful in the field of medicine, particularly in the development of therapeutics for neurodegenerative diseases.

1-8. (canceled)

9. A method of screening therapeutic candidates for treating tauopathy-related diseases, comprising:
the step of treating RAGE (receptor for advanced glycation end products) or cells expressing the RAGE with tau oligomers and at least one test substance;
the step of measuring the level of binding between the RAGE and the tau oligomers; and
the step of selecting a test substance that significantly reduces the level of binding compared to the control not treated with the test substance.

10. The method according to claim 9, wherein the tau oligomer is fluorescently labeled.

11. A method of screening therapeutic candidates for treating tauopathy-related diseases, comprising
the step of treating the cells expressing RAGE (receptor for advanced glycation end products) with a tauopathy-causing substance selected from the group consisting of i) neurofibrillary tangles (NFTs), ii) pathological brain tissue extracts from a tauopathic patient or a tauopathic model animal, and iii) tau oligomers, and at least one test substance;
the step of measuring the level of infection of tau protein into the cells treated with the test substance and the tauopathy-causing substance; and
the step of selecting a test substance that significantly reduces the level of infection of the tau protein into the cells compared to the control not treated with the test substance.

12. The method according to claim 11, wherein the cells are neurons or microglia.

13. The method according to claim 11, wherein the tau oligomer is fluorescently labeled.

14. The method according to claim 9, wherein the level of binding is measured using surface plasmon resonance (SPR), yeast two-hybrid assay, biolayer interferometry (BLI), immunoprecipitation (IP), or radioimmunoassay (RIA).

15. The method according to claim 9, wherein the test compound is a small compound, an extract of a microorganism, plant or animal, an antibody specific binding to RAGE or tau protein, and siRNA, shRNA or an antisense nucleotide that inhibits the expression of the RAGE.

16. The method according to claim 11, wherein the test compound is a substance that has been preliminarily screened by a method under in vitro conditions comprising the following steps:
the step of treating RAGE (receptor for advanced glycation end products) or cells expressing the RAGE with tau oligomers and at least one test substance;
the step of measuring the level of binding between the RAGE and the tau oligomers; and
the step of selecting a test substance that significantly reduces the level of binding compared to the control not treated with the test substance.


SEQUENCE LISTING (17 sequences)

SEQ ID NO: 1 - DNA, 1215 bp, synthetic construct
  misc_feature 1..1215: note = Human RAGE nucleotide sequence
  source 1..1215: mol_type = other DNA
atggctgccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttga

SEQ ID NO: 2 - protein, 404 aa, synthetic construct
  REGION 1..404: note = Human RAGE amino sequence
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 3 - DNA, 32 bp, synthetic construct
  misc_feature 1..32: note = P301L F
  source 1..32: mol_type = other DNA
gataatatcaaacacgtcctgggaggcggcag

SEQ ID NO: 4 - DNA, 32 bp, synthetic construct
  misc_feature 1..32: note = P301L R
  source 1..32: mol_type = other DNA
ctgccgcctcccaggacgtgtttgatattatc

SEQ ID NO: 5 - DNA, 32 bp, synthetic construct
  misc_feature 1..32: note = G82S F
  source 1..32: mol_type = other DNA
cgtgtccttcccaacagctccctcttccttcc

SEQ ID NO: 6 - DNA, 32 bp, synthetic construct
  misc_feature 1..32: note = G82S R
  source 1..32: mol_type = other DNA
ggaaggaagagggagctgttgggaaggacacg

SEQ ID NO: 7 - DNA, 30 bp, synthetic construct
  misc_feature 1..30: note = Cyto F
  source 1..30: mol_type = other DNA
ggggtcatcttgtggggggtaccagtcgac

SEQ ID NO: 8 - DNA, 30 bp, synthetic construct
  misc_feature 1..30: note = Cyto R
  source 1..30: mol_type = other DNA
gtcgactggtaccccccacaagatgacccc

SEQ ID NO: 9 - DNA, 30 bp, synthetic construct
  misc_feature 1..30: note = dV F
  source 1..30: mol_type = other DNA
cggaaggaagagggaacctactactgcccc

SEQ ID NO: 10 - DNA, 30 bp, synthetic construct
  misc_feature 1..30: note = dV R
  source 1..30: mol_type = other DNA
ggggcagtagtaggttccctcttccttccg

SEQ ID NO: 11 - DNA, 30 bp, synthetic construct
  misc_feature 1..30: note = dC1 F
  source 1..30: mol_type = other DNA
cagtgtgaagagccccttcccaggaatctg

SEQ ID NO: 12 - DNA, 30 bp, synthetic construct
  misc_feature 1..30: note = dC1 R
  source 1..30: mol_type = other DNA
cagattcctgggaaggggctcttcacactg

SEQ ID NO: 13 - DNA, 30 bp, synthetic construct
  misc_feature 1..30: note = dC2 F
  source 1..30: mol_type = other DNA
tatctcagggaggatctggatgggggctgt

SEQ ID NO: 14 - DNA, 30 bp, synthetic construct
  misc_feature 1..30: note = dC2 R
  source 1..30: mol_type = other DNA
acagcccccatccagatcctccctgagata

SEQ ID NO: 15 - DNA, 20 bp, synthetic construct
  misc_feature 1..20: note = targeted allele F
  source 1..20: mol_type = other DNA
agtgtcctcaggtcgggtga

SEQ ID NO: 16 - DNA, 24 bp, synthetic construct
  misc_feature 1..24: note = targeted allele R
  source 1..24: mol_type = other DNA
ccatctaagtgccagctaagggtc

SEQ ID NO: 17 - protein, 10 aa, synthetic construct
  REGION 1..10: note = RAGE antagonist peptide
ELKVLMEKEL